(19) United States
(12) Patent Application Publication
(10) Pub. No.: US 2025/0230456 A1
(43) Pub. Date: Jul. 17, 2025

(54) METHODS AND COMPOSITIONS FOR THE PRODUCTION OF UNREDUCED, NON-RECOMBINED GAMETES AND CLONAL OFFSPRING

(71) Applicants: Pioneer Hi-Bred International, Inc., Johnston, IA (US); UNIVERSITAET OF ZUERICH, Zuerich (CH); E.I. du PONT de NEMOURS AND COMPANY, Wilmington, DE (US)

(72) Inventors: Tim W. Fox, Des Moines, IA (US); Marc C. Albertsen, Grimes, IA (US); Mark E. Williams, Newark, DE (US); Shai J. Lawit, Urbandale, IA (US); Mark A. Chamberlin, Windsor Heights, IA (US); Ueli Grossniklaus, Zumikon (CH); Gion Arco Brunner, Horgen (CH); Nina Chumak, Adliswil (CH); Joana Bernardes De Asis, Birsfelden (CH); Frederique Pasquer, Horgen (CH)

(21) Appl. No.: 19/027,802

(22) Filed: Jan. 17, 2025

Related U.S. Application Data
(63) Continuation of application No. 15/571,510, filed on Nov. 3, 2017, now abandoned.
(60) Provisional application No. 62/157,687, filed on May 6, 2015.

(51) Int. Cl. C12N 15/82

(52) U.S. Cl. CPC C12N 15/8218; C12N 15/8241

(57) ABSTRACT

Methods and compositions used to prevent or decrease reduction, or reduction and recombination, during meiosis in female sporocyte, female gametophytes, or female gametes are described herein. The compositions and methods may include silencing elements and/or use gene-editing technology to decrease or inhibit Nrf4 expression levels or activity. Further provided are compositions and methods for the production of a plant that has ovules comprising non-reduced, or non-reduced and non-recombined, gametes, and/or seeds that contain clonal maternal embryos.

### CROSS-REFERENCE TO RELATED PATENT APPLICATIONS

This application claims benefit of U.S. Provisional Application No. 62/157,687, filed May 6, 2015, which is hereby incorporated herein by reference in its entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 37577_0001U1_Sequence_Listing.txt, created on Apr. 30, 2015, and having a size of 68,979 bytes and is filed concurrently with the specification. The sequence listing contained in this ASCII formatted document is part of the specification and is herein incorporated by reference in its entirety.

### FIELD OF THE DISCLOSURE

The present disclosure relates to the field of plant molecular biology, more particularly to plant female reproductive biology, and methods and compositions of altering reduction and recombination during female sporogenesis (meiosis in the ovule).

### BACKGROUND OF THE DISCLOSURE

Apomixis refers to asexual reproduction leading to the production of seeds without fertilization, that is to offspring which is genetically identical to the mother plant (Koltunow, et al., (1995) Plant Physiol. 108:1345-1352; Koltunow and Grossniklaus (2003) Ann. Rev. Plant Devl. 54: 547-74; Ravi, et al., (2008) Nature 451:1121-4). Apomixis is thus a reproductive process that bypasses female meiosis and syngamy to produce embryos identical to the maternal parent. Apomixis increases the opportunity for developing superior gene combinations and facilitates the rapid incorporation of desirable traits. Apomixis not only provides reproductive assurance, but also avoids a loss of heterozygosity in the offspring because the offspring maintains the parental genotype. Apomixis therefore avoids the effects of loss of vigor due to inbreeding and may additionally confer some advantages because of the heterosis effects.

At the species level, apomixis occurs in less than 1% of the species. Apomixis occurs in many wild species and in a few agronomically important species such as citrus and mango, but not in any of the major cereal crops (Eckhardt, (2003) Plant Cell 15:1449-1501). One form of apomixis is adventitious embryony, where embryos are formed directly out of somatic tissues within the ovules outside the embryo sac. Adventitious embryony usually occurs in parallel to normal sexual reproduction. A second form of apomixis is diplospory, which displaces sexual reproduction. In diplospory, an non-reduced egg cell is formed which then goes through a process called parthenogenesis (embryogenesis without fertilization) to form an embryo. A third form of apomixis is apopsory, which like adventitious embryony takes place in tissues outside the sexual embryo sac. Apospory, involves the formation of an asexual, non-reduced and non-recombined embryo sac that-as in diplospory-goes through parthenogenesis to form a clonal embryo. All three forms of apomixis rely on the production of an embryo without fertilization (parthenogenesis). Because apomixis offers the promise of the fixation and indefinite propagation of a desired genotype, there is a great deal of interest in engineering this ability to produce clonal seeds into crops, especially cereals (Spillane, et al., (2001) Sex. Plant Reprod. 14:179-87; Spillane, et al., (2004) Nat. Biotechnol. 22:687-91).

A molecular approach to engineer apomixis in commercial plant lines is highly desirable as a self-reproducing hybrid (SRH) system can result in a significant reduction in hybrid seed production costs. In other crops, such as soybean, it can result in the production, increase and sale of hybrid seeds, something which has not been done. There are three key components to apomixis: (1) avoidance of meiosis; (2) parthenogenic development of the embryo; and (3) achieving functional endosperm formation (Grossniklaus et al., (1998), in The Flowering of Apomixis: from Mechanisms to Genetic Engineering, eds. Savidan, Carman, Dresselhaus (CIMMYT, IRD, European Commission DG VI (FAIR), Mexico, DF), pp. 168-211; Grossniklaus, (2001), in Advances in Hybrid Rice Technology. Proceedings of the 3rd International Symposium on Hybrid Rice 1996, eds. Virmani, Siddiq, Muralidharan (International Rice Research Institute, Manila), pp. 187-211).

### BRIEF SUMMARY OF THE DISCLOSURE

The development of self-reproducing hybrids (SRH), or synthetic apomixis without reduction in meiosis by modifying Non-reductive in female4 (Nrf4) activity is disclosed herein.

Disclosed herein are methods of producing viable non-reduced, or viable non-reduced and non-recombined, gametes. The method comprises disrupting an endogenous Nrf4 gene. In an aspect, the method comprises disrupting an endogenous Nrf4 gene. in a plant or plant cell, wherein the plant cell is an ovule primordia, ovule, female gametophyte, or female gamete, thereby producing viable non-reduced, or viable non-reduced and non-recombined, gametes.

Provided herein is a nucleic acid construct that has a nucleotide sequence with an element that silences Nrf4 activity. Methods of decreasing Nrf4 activity in a plant is described. In some embodiments, the method includes decreasing the expression of one or more Nrf4 genes in a plant cell, for example, in an ovule primordia, ovule, female gametophyte, or female gamete. In some embodiments, the methods include disrupting an endogenous Nrf4 gene in a plant.

In other embodiments, methods of producing viable non-reduced, non-recombinant gametes are provided herein. A non-naturally occurring plant that has a disruption in an endogenous Nrf4 gene and develops ovules that include non-reduced, recombined gametes and/or non-reduced, non-recombined clonal gametes are described herein as well as seeds obtained from such a plant.

A further embodiment includes methods of maintaining heterozygosity in a progeny plant that has the same genotype as the parent plant by regenerating the progeny plant from a parent plant that has non-reduced, non-recombinant clonal gametes. In some examples, the endogenous Nrf4 gene is disrupted in the parent and progeny plant.

Also described herein are methods of expressing a sequence of interest in a plant or a plant cell. In some embodiments, the nucleotide sequence is expressed in an ovule-preferred manner in a plant. In some embodiments, the nucleotide sequence is expressed by a Nrf4 promoter, variant or fragment thereof.

### BRIEF DESCRIPTION OF THE DRAWINGS

FIG. 1. Ear phenotype of wild-type and nrf4/nrf4 mutant females crossed with a tetraploid male that produces diploid pollen. Plump kernels result from unreduced and shrunken kernels from reduced female gametophytes, respectively.

FIG. 2. Localization of Nrf4 transcripts in wild-type ovules of inbred line W22 determined by in situ hybridization. (FIG. 2A) Nrf4 transcript is abundant in the nucellus and integuments of early stage ovules. In the developing female sporocyte (megaspore mother cells—MMC) the Nrf4 signal was detected either at an intermediate (FIG. 2B) or high level (FIG. 2C). Images were taken with 10× magnification (FIG. 2A) or 40× magnification (FIG. 2B and FIG. 2C).

FIG. 3. Fluorescence image of a pair of maize florets of a young pre-fertilization ear (2.5 cm long) (FIG. 3A) and a young pre-fertilization ear (3.0 cm long) (FIG. 3B). These are T1 transgenic plants expressing the ZsGreen fluorescent protein (php56985) under the control of the ZmNRF4 promoter. (FIG. 3A) ZsGreen-positive expression is observed in the pre-meiotic ovules (arrows). Weak ZsGreen expression is also noted in the palea and lemma surrounding the ovule primordia, but not the glumes. (FIG. 3B) ZsGreen-positive expression is observed in the pre-meiotic ovules, their nucellus and surrounding ovular tissues. No expression was noted in the palea, lemma or glumes surrounding these slightly older ovules (compare with A).

### DETAILED DESCRIPTION

The present disclosure now will be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods for preventing or decreasing meiotic reduction in a female gametophyte or female gamete by modulating Nrf4 expression or activity is described herein. As used herein, “Nrf4 activity” refers to one or more of the following activities: (i) causing the reduction in meiosis during plant reproduction, for example, in an ovule, female sporocyte, female gametophyte, or female gamete, (ii) the recombination during the reduction in meiosis in a plant ovule, for example, in a female sporocyte. Nrf4 activity may occur in vitro or in vivo.

Manipulating Nrf4 expression and/or activity provides an opportunity to alter chromosome reduction during meiosis. Decreasing Nrf4 expression and/or activity in a plant may be used to create a plant with a non-reduced gamete, i.e. where the gamete retains a full chromosome set and does not have its genetic material reduced in half. Moreover, decreasing Nrf4 expression and/or activity in a plant may be used prevent recombination during sporogenesis. Provided herein are methods and compositions for producing plants having viable non-reduced, or non-reduced and non-recombined, gametes as well methods and compositions for maintaining heterozygosity in a progeny plant.

### II. Various Methods of Use

Methods and compositions for decreasing Nrf4 activity are provided. Disclosed herein are methods and compositions for decreasing Nrf4 activity in a female sporocyte, female gametophyte or female gamete in a plant. The method can include decreasing the expression level of one or more Nrf4 sequences, for example, gene, cDNA, polypeptide, in the female plant germline, for instance a female sporocyte, female gametophyte or female gamete. In some embodiments, methods and compositions are provided which employ a silencing element that decreases the level of expression and/or activity of Nrf4. Targets for Nrf4 polynucleotides include wild type Nrf4 polynucleotide or Nrf4 polypeptide sequences, Nrf4 variant polynucleotides, Nrf4 variant polypeptides, cognate promoter sequences, ortholog sequences, variants or fragments thereof. Silencing elements that decrease the expression of one or more of the Nrf4 target sequences and thereby decrease or inhibit the reduction and recombination in meiosis, e.g. Nrf4 activity, can be designed in view of these target polynucleotides.

Assays and techniques that measure reduction or recombination in meiosis include microscopic examination of spores, flow cytometry, crossing plants of various ploidy, and marker analysis, see, for example, Example 1 herein.

### III. Target Sequences

As used herein, a “target sequence” or “target polynucleotide” comprises any sequence that one desires to decrease the level of expression. In specific embodiments, decreasing the level of the target sequence in the female sporocyte, the female gametophyte or female gamete prevents or decreases reduction and/or recombination in meiosis. Non-limiting examples of target sequences include those in Table 1 and set forth in the sequence listing, any wild type Nrf4 polynucleotide or Nrf4 polypeptide sequences, Nrf4 variant polynucleotides, Nrf4 variant polypeptides, cognate promoter sequences, ortholog sequences, variants or fragments thereof. As exemplified elsewhere herein, decreasing the level of expression of one or more of these target sequences results in the non-reduction and non-recombination during meiosis in the plant female sporocyte, female gametophyte or female gamete.

### IV. Silencing Elements

By “silencing element” is intended a polynucleotide that is capable of reducing or eliminating the level or expression of a target polynucleotide or the polypeptide encoded thereby. The silencing element employed can decrease or eliminate the expression level of the target sequence by influencing the level of the target RNA transcript or, alternatively, by influencing translation and thereby affecting the level of the encoded polypeptide. Methods to assay for functional silencing elements that are capable of decreasing or eliminating the level of a sequence of interest are disclosed elsewhere herein. A single polynucleotide employed in the methods can comprise one or more silencing elements to the same or different target polynucleotides. The silencing element can be produced in vivo (i.e., in a host cell such as a plant or microorganism) or in vitro.

In specific embodiments, the target sequence is endogenous to the plant. In other embodiments, while the silencing element regulates non-reduction and non-recombination of meiosis, preferably the silencing element has no effect on the parts of the plant that do not constitute the female germline (female sporocyte, female gametophyte, and female gamete).

As discussed in further detail below, silencing elements can include, but are not limited to, a sense suppression element, an antisense suppression element, a double stranded RNA, a siRNA, an amiRNA, a miRNA, or a hairpin suppression element. Non-limiting examples of silencing elements that can be employed to decrease expression of these target sequences or additionally sequences targeting genes involved in recombination comprise fragments and variants of the sense or antisense sequence or consists of the sense or antisense sequence of any of the sequences in Table 1, set forth in the sequence listing, wild type Nrf4 polynucleotide or Nrf4 polypeptide sequences, Nrf4 variant polynucleotides, Nrf4 variant polypeptides, cognate promoter sequences, ortholog sequences, variants or fragments thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. As discussed in further detail below, enhancer suppressor elements can also be employed in conjunction with the silencing elements disclosed herein.

By “decreases” or “decreasing” the expression level of a polynucleotide or a polypeptide encoded thereby is intended to mean, the polynucleotide or polypeptide level of the target sequence is lower than the polynucleotide level or polypeptide level of the same target sequence in an appropriate control in which the silencing element is not introduced. In particular embodiments, decreasing the polynucleotide level and/or the polypeptide level of the target sequence results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control. Methods to assay for the level of the RNA transcript, the level of the encoded polypeptide, or the activity of the polynucleotide or polypeptide are discussed elsewhere herein.

### A. Sense Suppression/Cosuppression

In some embodiments of the disclosure, decreasing expression of a Nrf4 polypeptide may be obtained by sense suppression or cosuppression. For cosuppression, an expression cassette is designed to express an RNA molecule corresponding to all or part of a messenger RNA encoding a polypeptide in the “sense” orientation. Over expression of the RNA molecule may result in decreased expression of the native gene. Accordingly, multiple plant lines transformed with the cosuppression expression cassette are screened to identify those that show the desired degree of inhibition of polypeptide expression.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it allows for the suppression of the targeted sequence. The sense suppression element can be, for example, 15, 16, 17, 18 19, 20, 22, 25, 30, 50, 100, 150, 200, 250, 300, 350, 400, 450, 500, 600, 700, 900, 1000, 1100, 1200, 1300 nucleotides or longer of the target polynucleotides set forth in any of SEQ ID NO:3-8, 10, 23, 24, 17, 18, 22, 28 or 29. In other embodiments, the sense suppression element can be, for example, about 15-25, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides set forth in any of SEQ ID NO:3-8, 10, 23, 24, 17, 18, 22, 28 or 29.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In some embodiments where the polynucleotide comprises all or part of the coding region for the polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

### B. Antisense Suppression

In some embodiments of the disclosure, inhibition of the expression of the polypeptide may be obtained by antisense suppression. For antisense suppression, the expression cassette is designed to express an RNA molecule complementary to all or part of a messenger RNA encoding the polypeptide. Over expression of the antisense RNA molecule may result in decreased expression of the target gene. Accordingly, multiple plant lines transformed with the antisense suppression expression cassette are screened to identify those that show the desired degree of inhibition of polypeptide expression.

The polynucleotide for use in antisense suppression may correspond to all or part of the complement of the sequence encoding the polypeptide, all or part of the complement of the 5′ and/or 3′ untranslated region of the target transcript or all or part of the complement of both the coding sequence and the untranslated regions of a transcript encoding the polypeptide. In addition, the antisense polynucleotide may be fully complementary (i.e., 100% identical to the complement of the target sequence) or partially complementary (i.e., less than 100% identical to the complement of the target sequence) to the target sequence. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 20, 22, 25, 50, 100, 200, 300, 400, 450 nucleotides or greater of the sequence set forth in any of SEQ ID NO:3-8, 10, 23, 24, 17, 18, 22, 28 or 29may be used. Methods for using antisense suppression to inhibit the expression of endogenous genes in plants are described, for example, in Liu et at (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

### C. Double-Stranded RNA Interference

In some embodiments of the disclosure, inhibition of the expression of a polypeptide may be obtained by double-stranded RNA (dsRNA) interference. A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions mediate the decrease of expression of a target sequence, for example, by mediating RNA interference “RNAi” or gene silencing in a sequence-specific manner. The dsRNA is capable of decreasing or eliminating the level or expression of a target polynucleotide or the polypeptide, for example, Nrf4.

The dsRNA can decrease or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al., (2004) Science 303:672-676; Pal-Bhadra et al., (2004) Science 303:669-672; Allshire (2002) Science 297:1818-1819; Volpe et al., (2002) Science 297:1833-1837; Jenuwein (2002) Science 297:2215-2218; and Hall et al., (2002) Science 297:2232-2237. Methods to assay for functional dsRNA that are capable of decreasing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules may be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the desired degree of inhibition of polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

### D. Hairpin RNA Interference and Intron-Containing Hairpin RNA Interference

In some embodiments of the disclosure, inhibition of the expression of a polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38, and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene whose expression is to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731; and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7; and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295; and US Patent Application Publication Number 2003/0180945, each of which is herein incorporated by reference.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments, a domain of the silencing element shares sufficient homology to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al., (2003) J. Biol. Chem. 278:7108-7118 and Yang et al., (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci. USA 99:13659-13662; Aufsaftz, et al., (2002) Proc. Natl. Acad. Sci. 99:16499-16506; Sijen, et al., Curr. Biol. (2001) 11:436-440, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al., (2002) Proc. Natl. Acad. Sci. 99:16499-16506 and Mette et al., (2000) EMBO J. 19:5194-5201.

### E. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the polypeptide). Methods of using amplicons to inhibit the expression of endogenous plant genes are described, for example, in Angell and Baulcombe, (1997) EMBO J. 16:3675-3684; Angell and Baulcombe, (1999) Plant J. 20:357-362; and U.S. Pat. No. 6,646,805, each of which is herein incorporated by reference.

### F. Ribozymes

In some embodiments, the polynucleotide expressed by the expression cassette of the disclosure is a catalytic RNA or has ribozyme activity specific for the messenger RNA of the polypeptide. Thus, the polynucleotide causes the degradation of the endogenous messenger RNA, resulting in decreased expression of the polypeptide. This method is described, for example, in U.S. Pat. No. 4,987,071, herein incorporated by reference.

### G. Small Interfering RNA or Micro RNA

In some embodiments of the disclosure, inhibition of the expression of a polypeptide may be obtained by RNA interference by expression of a gene encoding a micro RNA (miRNA) or short-interfering RNA (siRNA) (Meister and Tuschl (2004) Nature 431:343-349 and Bonetta et al., (2004) Nature Methods 1:79-86). miRNAs are regulatory agents consisting of about 22 ribonucleotides. miRNA are highly efficient at inhibiting the expression of endogenous genes. See, for example Palatnik et al., (2003) Nature 425:257-263, herein incorporated by reference. The miRNA can be an “artificial miRNA” or “amiRNA” which comprises a miRNA sequence that is synthetically designed to silence a target sequence.

For miRNA interference, the expression cassette is designed to express an RNA molecule that is modeled on an endogenous miRNA gene. For example, the miRNA gene encodes an RNA that forms a hairpin structure containing a 22-nucleotide sequence that is complementary to another endogenous gene (target sequence). In some embodiments, the 22-nucleotide sequence is selected from a transcript sequence from a Nrf4 gene and contains 22 nucleotides of the Nrf4 gene in sense orientation and 21 nucleotides of a corresponding antisense sequence that is complementary to the sense sequence. In some embodiments, in addition to targeting Nrf4, genes involved in recombination may also be targeted. Accordingly, in some embodiments, the 22-nucleotide sequence is selected from a transcript sequence from a gene involved in recombination and contains 22 nucleotides of the gene involved in recombination in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

The heterologous polynucleotide being expressed need not form the dsRNA by itself, but can interact with other sequences in the plant cell to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then decrease the level of expression of the gene or genes to be silenced. See, for example, US Application Publication 2007-0130653, entitled “Methods and Compositions for Gene Silencing”, herein incorporated by reference. The construct can be designed to have a target for an endogenous miRNA or, alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

While the various target sequences disclosed herein can be used to design any silencing element that encodes a miRNA, non-limiting examples of such miRNA constructs include SEQ ID NOS: 33-37; or active variants or fragments thereof.

### H. Polypeptide-Based Inhibition of Gene Expression

In one embodiment, the polynucleotide encodes a zinc finger protein that binds to a gene encoding a polypeptide, resulting in decreased expression of the gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 2003/0037355, each of which is herein incorporated by reference.

### I. Polypeptide-Based Inhibition of Protein Activity

In some embodiments of the disclosure, the polynucleotide encodes an antibody that binds to at least one polypeptide and decreases the activity of the polypeptide. In another embodiment, the binding of the antibody results in increased turnover of the antibody complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### V. Gene Disruption

In some embodiments of the present disclosure, the activity of a polypeptide is decreased or eliminated by disrupting the gene encoding the polypeptide. The gene encoding the polypeptide may be disrupted by any method known in the art, for example, by genome editing, transposon tagging or mutagenizing plants using random or targeted mutagenesis and selecting for plants that have decreased activity.

### A. Genome Editing and Induced Mutagenesis

In some embodiments, the target, for example, Nrf4 can be modified using gene editing technology, including without limitation double-strand-break-inducing agent, such as but not limited to a CRISPR-Cas guideRNA or other polynucleotide-guided double strand break reagent, a Zinc Finger endonuclease, a meganuclease, or a TALEN endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times, also referred to as CRISPR-repeats) which are partially palindromic. The repeated sequences (usually specific to a species) are interspaced by variable sequences of constant length (typically 20 to 58 bp depending on the CRISPR locus (WO2007/025097 published Mar. 1, 2007).

CRISPR loci were first recognized in Escherichia coli (Ishino et al., (1987) J. Bacteriol. 169:5429-5433; Nakata et al., (1989) J. Bacteriol. 171:3553-3556). Similar interspersed short sequence repeats have been identified in Haloferax mediterranei, Streptoccoccus pyogenes, Anabaena, and Mycobacterium tuberculosis (Groenen et al., (1993) Mol. Microbiol. 10:1057-1065; Hoe et al., (1999) Emerg. Infect. Dis. 5:254-263; Masepohl et al., (1996) Biochim. Biophys. Acta 1307:26-30; Mojica et al. (1995) Mol. Microbiol. 17:85-93). The CRISPR loci differ from other simple sequence repeats (SSRs) by the structure of the repeats, which have been termed short regularly spaced repeats (SRSRs) (Janssen et al., (2002) J. Integ. Biol. 6:23-33; Mojica et al., (2000) Mol. Microbiol. 36:244-246). The repeats are short elements that occur in clusters, which are always regularly spaced by variable sequences of constant length (Mojica et al., (2000) Mol. Microbiol. 36:244-246).

Cas gene relates to a gene that is generally coupled, associated or close to, or in the vicinity of flanking CRISPR loci. The terms “Cas gene”, “CRISPR-associated (Cas) gene” are used interchangeably herein. A comprehensive review of the Cas protein family is presented in Haft et al., (2005) PLoS Comput Biol 1: e60. As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (U.S. Provisional Application No. 62/023,239, filed Jul. 11, 2014). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

The Cas endonuclease gene can encode Cas9 endonuclease, or a functional fragment thereof, such as but not limited to the Cas9 genes listed in SEQ ID NOs: 462, 474, 489, 494, 499, 505, and 518 of WO2007/025097, published Mar. 1, 2007. The Cas endonuclease gene can be a plant, maize or soybean optimized Cas9 endonuclease, such as but not limited to a plant codon optimized Streptoccoccus pyogenes Cas9 gene that can recognize any genomic sequence of the form N(12-30)NGG. The Cas endonuclease can be introduced directly into a cell by any method known in the art, for example, but not limited to, transient introduction methods, transfection, and/or topical application.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site (U.S. Provisional Application No. 62/023,239, filed Jul. 11, 2014). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited to, Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”. The guide polynucleotide can be a double molecule (also referred to as duplex guide polynucleotide), comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In one embodiment of the disclosure, the single guide RNA comprises a CRNA or cRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide) that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to, a GAAA tetraloop seqence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide polynucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide, a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

### B. Transposon Tagging

In one embodiment of the disclosure, transposon tagging is used to decrease or eliminate the activity of one or more polypeptides. Transposon tagging comprises inserting a transposon within an endogenous gene in the pathway to decrease or eliminate expression of the polypeptide.

In this embodiment, the expression of one or more polypeptides is decreased or eliminated by inserting a transposon within a regulatory region or coding region of the gene encoding the polypeptide. A transposon that is within an exon, intron, 5′ or 3′ untranslated sequence, a promoter or any other regulatory sequence of a gene may be used to decrease or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner et al., (2000) Plant J. 22:265-274; Phogat, et al., (2000) J. Biosci. 25:57-63; Walbot, (2000) Curr. Opin. Plant Biol. 2:103-107; Gai et al., (2000) Nucleic Acids Res. 28:94-96; Fitzmaurice et al., (1999) Genetics 153:1919-1928. In addition, the TUSC process for selecting Mu insertions in selected genes has been described in Bensen et al., (1995) Plant Cell 7:75-84; Mena et al., (1996) Science 274:1537-1540; and U.S. Pat. No. 5,962,764, each of which is herein incorporated by reference.

### C. Mutant Plants With Decreased Activity

Additional methods for decreasing or eliminating the expression of endogenous genes in plants are also known in the art and may be similarly applied to the instant These methods include other forms of mutagenesis, such as ethyl disclosure. methanesulfonate-induced mutagenesis, deletion mutagenesis, and fast neutron deletion mutagenesis used in a reverse genetics sense (with PCR) to identify plant lines, in which the endogenous gene has been mutated or deleted. For examples of these methods see Ohshima et al., (1998) Virology 243:472-481; Okubara et al., (1994) Genetics 137:867-874; and Quesada et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See McCallum et al., (2000) Nat. Biotechnol. 18:455-457, herein incorporated by reference.

Mutations may impact gene expression or interfere with the activity of an encoded Nrf4 protein. Insertional mutations in gene exons usually result in null-mutants. Mutations in conserved residues are particularly effective in inhibiting the activity of the encoded protein. Conserved residues of plant polypeptides suitable for mutagenesis with the goal to eliminate activity have been described. Such mutants may be isolated according to well-known procedures and mutations in different Nrf4 loci may be stacked by genetic crossing. See, for example, Gruis et al., (2002) Plant Cell 14:2863-2882.

In another embodiment of this disclosure, dominant mutants may be used to trigger RNA silencing due to gene inversion and recombination of a duplicated gene locus. See, for example, Kusaba et al., (2003) Plant Cell 15:1455-1467.

The disclosure encompasses additional methods for decreasing or eliminating the activity of one or more target polypeptides. Examples of other methods for altering or mutating a genomic nucleotide sequence in a plant are known in the art and include, but are not limited to, the use of RNA: DNA vectors, RNA: DNA mutational vectors, RNA: DNA repair vectors, mixed-duplex oligonucleotides, self-complementary RNA: DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984, each of which are herein incorporated by reference. See also WO 1998/49350, WO 1999/07865, WO 1999/25821, and Beetham et al., (1999) Proc. Natl. Acad. Sci. USA 96:8774-8778, each of which is herein incorporated by reference.

### VI. Expression Constructs

Methods and compositions are provided to modify the level of expression or activity of a Nrf4 polypeptide in a plant cell, for example, in an ovule primordia, ovule, female sporocyte, female gametophyte, or female gamete. In some embodiments, a plant cell is transformed with a DNA construct or expression cassette for expression of at least one silencing element. In specific embodiments, modulation of Nrf4 expression level and/or activity of the Nrf4 polypeptide promotes non-reduction, or non-reduction and non-recombination, during meiosis resulting in the production of non-reduced, or non-reduced and non-recombined, female gametes. Such methods and compositions can employ an expression construct comprising an element that when expressed decreases Nrf4 polynucleotide and/or Nrf4 polypeptide expression level or activity and is operably linked to a promoter functional in a plant cell. In certain embodiments, the promoter is a female sporogenesis-related promoter, in particular a promoter expressing in ovule primordia or ovule tissue, including but not limited to an Nrf4 promoter.

The expression cassette can include 5′ and 3′ regulatory sequences operably linked to a polynucleotide of interest, e.g. a silencing element, or an active variant or fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest, for example, a silencing element. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional polynucleotides can be provided on multiple expression cassettes. Such an expression cassette is provided with a plurality of restriction sites and/or recombination sites for insertion of the element to be under the transcriptional regulation of the promoter. The expression cassette may additionally contain selectable marker genes.

In some embodiments, the expression cassette will include in the 5′-3′ direction of transcription an ovule-specific promoters, ovule-preferred promoters, female-gametophyte specific promoters, female-gametophyte preferred promoters, female-gamete-specific promoters, female-gamete-preferred promoters, a female sporogenesis-related promoter or an active variant or fragment thereof, a silencing element and a transcriptional and translational termination region (i.e., termination region) functional in the host cell (i.e., the plant). The regulatory regions and/or the silencing elements may be heterologous to the host cell, e.g. plant cell, or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence. In an aspect, the promoter is a heterologous promoter.

The termination region may be native with the transcriptional initiation region, may be native with the operably linked silencing element or with the ovule tissue-preferred promoter sequences, may be native with the plant host, or may be derived from another source (i.e., foreign or heterologous) to the promoter, the silencing element, the plant host, or any combination thereof. Convenient termination regions are available from the Ti-plasmid of Agrobacterium tumefaciens, such as the octopine synthase and nopaline synthase gene termination regions. See also Guerineau et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391, and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The constructs or expression cassettes may additionally contain 5′ leader sequences. Such leader sequences can act to enhance translation. Translation leaders are known in the art and include: picornavirus leaders, for example, EMCV leader (Encephalomyocarditis Virus 5′ noncoding region) (Elroy-Stein et al., (1989) Proc. Natl. Acad. Sci. USA 86:6126-6130); potyvirus leaders, for example, TEV leader (Tobacco Etch Virus) (Gallie et al., (1995) Gene 165:233-238), MDMV leader (Maize Dwarf Mosaic Virus) (Johnson et al., (1986) Virology 154:9-20), and human immunoglobulin heavy-chain binding protein (BiP) (Macejak et al., (1991) Nature 353:90-94); untranslated leader from the coat protein mRNA of Alfalfa Mosaic Virus (AMV RNA 4) (Jobling et al., (1987) Nature 325:622-625); tobacco mosaic virus (TMV) leader (Gallie et al., (1989) in Molecular Biology of RNA, ed. Cech (Liss, New York), pp. 237-256), and Maize Chlorotic Mottle Virus (MCMV) leader (Lommel et al., (1991) Virology 81:382-385). See also Della-Cioppa et al., (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, substitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers, such as β-galactosidase and fluorescent proteins, for example green fluorescent protein (GFP) (Su et al., (2004) Biotechnol Bioeng 85:610-9 and Fetter et al., (2004) Plant Cell 16:215-28), cyan florescent protein (CYP) (Bolte et al., (2004) J. Cell Science 117:943-54 and Kato et al., (2002) Plant Physiol 129:913-42), yellow florescent protein (PhiYFP™ from Evrogen, see Bolte et al., (2004) J. Cell Science 117:943-54), and red fluorescent protein (DsRED, see Baird et al., (2000) Proc. Natl. Acad. Sci. USA. 97:11984-11989). For additional selectable markers, see, generally, Yarranton, (1992) Curr. Opin. Biotech 3:506-511; Christopherson et al., (1992) Proc. Natl. Acad. Sci. USA 89:6314-6318; Yao et al., (1992) Cell 71:63-72; Reznikoff, (1992) Mol. Microbiol. 6:2419-2422; Barkley et al., (1980) in The Operon, pp. 177-220; Hu et al., (1987) Cell 48:555-566; Brown et al., (1987) Cell 49:603-612; Figge et al., (1988) Cell 52:713-722; Deuschle et al., (1989) Proc. Natl. Acad. Aci. USA 86:5400-5404; Fuerst et al., (1989) Proc. Natl. Acad. Sci. USA 86:2549-2553; Deuschle et al., (1990) Science 248:480-483; Gossen, (1993) Ph.D. Thesis, University of Heidelberg; Reines et al., (1993) Proc. Natl. Acad. Sci. USA 90:1917-1921; Labow et al., (1990) Mol. Cell. Biol. 10:3343-3356; Zambretti et al., (1992) Proc. Natl. Acad. Sci. USA 89:3952-3956; Baim et al., (1991) Proc. Natl. Acad. Sci. USA 88:5072-5076; Wyborski et al., (1991) Nucleic Acids Res. 19:4647-4653; Hillenand-Wissman, (1989) Topics Mol. Struc. Biol. 10:143-162; Degenkolb et al., (1991) Antimicrob Agents Chemother 35:1591-1595; Kleinschmidt et al., (1988) Biochemistry 27:1094-1104; Bonin, (1993) Ph.D. Thesis, University of Heidelberg; Gossen et al., (1992) Proc. Natl. Acad. Sci. USA 89:5547-5551; Oliva et al., (1992) Antimicrob Agents Chemother 36:913-919; Hlavka et al., (1985) Handbook of Experimental Pharmacology, Vol. 78 (Springer-Verlag, Berlin); Gill et al., (1988) Nature 334:721-724. Such disclosures are herein incorporated by reference. The above list of selectable marker genes is not meant to be limiting. Any selectable marker gene can be used.

It is further recognized that various expression constructs other than the Nrf4 silencing expression construct are described herein. For example, expression constructs having silencing elements for genes involved in recombination and/or sequences encoding marker sequences are also described herein. One of skill will understand how to apply the language discussed above, to any expression construct.

### VII. Nrf4 Sequences

Disclosed herein in Table 1 below and elsewhere are isolated or substantially purified nucleic acid molecules or protein compositions of wild type Nrf4 polynucleotide or Nrf4 polypeptide sequences, Nrf4 variant polynucleotides, Nrf4 variant polypeptides, cognate promoter sequences, ortholog sequences, variants or fragments thereof that can be modified to effect non-reduction and non-recombination during meiosis in a plant female sporocyte, female gametophyte, or female gamete.

TABLE 1

| | | | | POLY- |
|---|---|---|---|---|
| | | | | NUCLEO- |
| | | | | TIDE/ |
| | | | | POLY- |
| SEQ | | DE- | | PEPTIDE |
| ID. | NAME | SCRIPTION | SPECIES | (PN/PP) |
| SEQ ID | NRF4 | protein | Zea mays | PP |
| NO: 1 | | | | |
| SEQ ID | NRF4-ALT | Protein- | Zea mays | PP |
| NO: 2 | | Alternative | | |
| | | splicing | | |
| SEQ ID | NRF4 | mRNA | Zea mays | PN |
| NO: 3 | | | | |
| SEQ ID | NRF4 | CDS | Zea mays | PN |
| NO: 4 | | | | |
| SEQ ID | NRF4-ALT | CDS- | Zea mays | PN |
| NO: 5 | | alternative | | |
| | | splicing | | |
| SEQ ID | NRF4 | 1625 bp 5′ of | Zea mays | PN |
| NO: 6 | | transcription | | |
| | | start | | |
| | | (promoter) | | |
| SEQ ID | NRF4 | genomic | Zea mays | PN |
| NO: 7 | | | | |
| SEQ ID | Bradi1g19285.1 | CDS | Brachypodium | PN |
| NO: 8 | | | distachyon | |
| SEQ ID | Bradi1g19285.1 | protein | Brachypodium | PP |
| NO: 9 | | | distachyon | |
| SEQ ID | Bradi1g19285.1 | genomic | Brachypodium | PN |
| NO: 10 | | | distachyon | |
| SEQ ID | Bradi1g19285.1 | promoter | Brachypodium | PN |
| NO: 11 | | | distachyon | |
| SEQ ID | Sobic | CDS | Sorghum | PN |
| NO: 12 | 002G400700.1 | | bicolor | |
| SEQ ID | Sobic | genomic | Sorghum | PN |
| NO: 13 | 002G400700 | | bicolor | |
| SEQ ID | Sobic | protein | Sorghum | PP |
| NO: 14 | 002G400700.1 | | bicolor | |
| SEQ ID | Sobic | promoter | Sorghum | PN |
| NO: 15 | 002G400700.1 | | bicolor | |
| SEQ ID | Pavirv00009878m | protein | Panicum | PP |
| NO: 16 | | | virgatum | |
| SEQ ID | Pavirv00009878m | CDS | Panicum | PN |
| NO: 17 | | | virgatum | |
| SEQ ID | Pavirv00009878m | genomic | Panicum | PN |
| NO: 18 | | | virgatum | |
| SEQ ID | Pavirv00009878m | promoter | Panicum | PN |
| NO: 19 | | | virgatum | |
| SEQ ID | Si030723 | protein | Setaria italica | PP |
| NO: 20 | | | | |
| SEQ ID | Si030723 | promoter | Setaria italica | PN |
| NO: 21 | | (1.7kb) | | |
| SEQ ID | NRF4 | CDS with | Oryza sativa | PN |
| NO: 22 | orthologue | introns from | | |
| | | AP005184 and | | |
| | | AP005182 | | |
| SEQ ID | NRF4 | Promoter | Oryza sativa | PN |
| NO: 23 | orthologue | (1.7kb): from | | |
| | promoter | AP005184 and | | |
| | Chr7.fgenesh. | AP005182 | | |
| | mRNA.4040 | | | |
| SEQ ID | protein | protein | Oryza sativa | PP |
| NO: 24 | | | | |
| SEQ ID | Protein- | protein | Oryza sativa | PP |
| NO: 25 | putative from | | | |
| | Indica | | | |
| SEQ ID | Taes_2DS_DE | Promoter | Triticum | PN |
| NO: 26 | 9475523 | (954bp) | aestivum | |
| SEQ ID | Taes_2DS_DE | protein | Triticum | PP |
| NO: 27 | 9475523 | | aestivum | |
| SEQ ID | scaffold:IWGSP1: | Genomic | Triticum | PN |
| NO: 28 | IWGSC_CSS_ | | aestivum | |
| | 2DS_scaff_ | | | |
| | 5389501: | | | |
| | 1958:5178:−1 | | | |
| SEQ ID | scaffold:IWGSP1: | CDS | Triticum | PN |
| NO: 29 | IWGSC_CSS_ | | aestivum | |
| | 2DS_scaff_ | | | |
| | 5389501: | | | |
| | 1958:5178:−1 | | | |
| SEQ ID | PHN130859- | oligonucleotide | Artificial | PN |
| NO: 30 | Muint26 | | sequence | |
| SEQ ID | PHN130859- | oligonucleotide | Artificial | PN |
| NO: 31 | Muint26- | | sequence | |
| | PE2.0 | | | |
| SEQ ID | PE1.0 | oligonucleotide | Artificial | PN |
| NO: 32 | | | sequence | |
| SEQ ID | ZM MIRNA | oligonucleotide | Artificial | PN |
| NO: 33 | 5END | | sequence | |
| | PRECURSOR | | | |
| | 396H | | | |
| SEQ ID | ZM-NRF4A | oligonucleotide | Artificial | PN |
| NO: 34 | | | sequence | |
| SEQ ID | ZM MRNA | oligonucleotide | Artificial | PN |
| NO: 35 | PRECURSOR | | sequence | |
| | 396H | | | |
| SEQ ID | ZM-NRF4A | oligonucleotide | Artificial | PN |
| NO: 36 | 396H STAR | | sequence | |
| SEQ ID | ZM MIRNA | oligonucleotide | Artificial | PN |
| NO: 37 | 3END | | sequence | |
| | PRECURSOR | | | |
| | 396H | | | |

As used herein, an “isolated” or “purified” polynucleotide or polypeptide or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5% or 1% (by dry weight) of contaminating protein. When the polypeptide of the disclosure or biologically active portion thereof is recombinantly produced, optimally culture medium represents less than about 30%, 20%, 10%, 5% or 1% (by dry weight) of chemical precursors or non-protein-of-interest chemicals.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

A “control” or “control plant” or “control plant cell” provides a reference point for measuring changes in phenotype of the subject plant or plant cell, and may be any suitable plant or plant cell. A control plant or plant cell may comprise, for example: (a) a wild-type or native plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell which is genetically identical to the subject plant or plant cell but which is not exposed to the same treatment (e.g., herbicide treatment) as the subject plant or plant cell; (e) the subject plant or plant cell itself, under conditions in which the gene of interest is not expressed or (f) a plant or plant cell which is a non-transformed with a construct containing a silencing element.

### VIII. Sequence Identity

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins et al., (1988) Gene 73:237-244 (1988); Higgins et al., (1989) CABIOS 5:151-153; Corpet et al., (1988) Nucleic Acids Res. 16:10881-90; Huang et al., (1992) CABIOS 8:155-65 and Pearson, et al. (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul et al., (1997) supra. When utilizing BLAST, Gapped BLAST and PSI-BLAST, the default parameters of the respective programs (e.g., BLASTN for nucleotide sequences, BLASTX for proteins) can be used. See the web site of the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, 70%, 80%, 90% and at least 95%.

### IX. Promoters

Various types of promoters can be employed in the methods and compositions provided herein. Promoters can drive expression in a manner that is cell-type-preferred, cell-type-specific, tissue-preferred or tissue-specific. Examples of promoters under developmental control include promoters that preferentially initiate transcription in certain tissues, such as leaves, roots, seeds or ovules. Such promoters are referred to as “tissue preferred”. Promoters which initiate transcription only in certain tissue are referred to as “tissue specific”. A “cell type” preferred promoter primarily drives expression in certain cell types in one or more organs, for example, vascular cells in roots, leaves or ovules. An “inducible” or “repressible” promoter is a promoter which is under environmental or chemical control. Examples of environmental conditions that may affect transcription by inducible promoters include anaerobic conditions or the presence of light. Tissue specific, tissue preferred, cell type specific, cell type preferred and inducible promoters constitute the class of “non-constitutive” promoters. A “constitutive” promoter is a promoter that is active under most environmental conditions and in all tissues throughout development.

Non-limiting examples of constitutive promoters include, for example, the core promoter of the Rsyn7 promoter and other constitutive promoters disclosed in WO 1999/43838 and U.S. Pat. No. 6,072,050; the core CaMV 35S promoter (Odell et al., (1985) Nature 313:810-812); the rice actin promoter (McElroy et al., (1990) Plant Cell 2:163-171); he ubiquitin promoter (Christensen et al., (1989) Plant Mol. Biol. 12:619-632 and Christensen et al., (1992) Plant Mol. Biol. 18:675-689); the pEMU promoter (Last et al., (1991) Theor. Appl. Genet. 81:581-588); the MAS promoter (Velten et al., (1984) EMBO J. 3:2723-2730); the ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

One of skill will recognize that the sequences encoding elements or polypeptides can be placed into an expression cassette. Expression cassettes are discussed elsewhere herein. Any promoter of interest can be operably linked to the sequence encoding the elements or polypeptides, including, for example, constitutive promoters, tissue-preferred promoters, tissue-specific promoters, female sporocyte-specific promoters, female sporocyte-preferred promoters, female gametophyte-specific promoters, female gametophyte-preferred promoters, female gamete-specific promoters, female gamete-preferred promoters, ovule tissue-preferred promoters, an ovule tissue-preferred promoter that is active in at least one non-gametophyte tissue in a plant ovule, seed-preferred, embryo-preferred and/or endosperm preferred promoters. Many such promoters have been described elsewhere herein or are known in the art.

By “promoter” is intended a regulatory region of DNA usually comprising a TATA box capable of directing RNA polymerase II to initiate RNA synthesis at the appropriate transcription initiation site for a particular polynucleotide sequence. A promoter may additionally comprise other recognition sequences generally positioned upstream or 5′ to the TATA box, referred to as upstream promoter elements, which influence the transcription initiation rate. The promoter sequences disclosed herein regulate (i.e., activate) transcription from the promoter region.

In some embodiments, the methods and compositions include isolated polynucleotides comprising Nrf4 promoters. The Nrf4 promoter nucleotide sequences include those set forth in SEQ ID NOS: 11, 15, 19, 21, 23 and 26, active variants and fragments thereof. In one embodiment, an expression construct includes any of the polynucleotides set forth in SEQ ID NOS: 11, 15, 19, 21, 23 and 26 operably linked to the polynucleotide of interest or any polynucleotide having at least 95%, 96%, 97%, 98% or 99% sequence identity to the sequence set forth in SEQ ID NO: 11, 15, 19, 21, 23 and 26, wherein said polynucleotide retains the ability to direct expression of an operably linked polynucleotide in ovule developmental tissue prior to the initiation of sporogenesis and/or prior to or during meiosis.

Fragments and variants of each of the promoter nucleotide sequences set forth in SEQ ID NOS: 11, 15, 19, 21, 23 and 26 are further provided herein. Fragments of a promoter polynucleotide may retain biological activity and, hence, retain transcriptional regulatory activity. Thus, fragments of a promoter nucleotide sequence may range from at least about 20 nucleotides, about 50 nucleotides, about 100 nucleotides, and up to the full-length polynucleotide of the disclosure. Thus, a fragment of promoter that drives expression during female sporogenesis, a female sporogenesis-related promoter, such as a promoter that expresses in ovule developmental tissue prior to and/or during meiosis, may encode a biologically active portion of a female sporogenesis-related promoter. A biologically active portion of a female sporogenesis-related promoter polynucleotide can be prepared by isolating a portion of one of the female sporogenesis-related promoter polynucleotides, and assessing the activity of the portion of the megasporogenesis-related promoter. Polynucleotides that are fragments of a female sporogenesis-related promoter polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, 2000, nucleotides or up to the number of nucleotides present in a full-length female sporogenesis-related promoter polynucleotide disclosed herein.

For a promoter polynucleotide, a variant comprises a deletion and/or addition of one or more nucleotides at one or more internal sites within the native polynucleotide and/or a substitution of one or more nucleotides at one or more sites in the native polynucleotide. Generally, variants of a particular ovule tissue-preferred promoter will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Thus, any of the Nrf4 promoters, variants, and fragments thereof may be utilized to regulate a number of genes and developmental processes during female sporogenesis. The Nrf4 promoter may be used to ectopically express an RNA resulting in protein production, silencing, gene modification, modification of other RNA(s), or catalysis of a reaction.

As discussed herein, various promoters can be employed in the methods and compositions provided herein, including: promoters to express sequences encoding elements that silence or reduce Nrf4 activity and/or recombination activity. An Nrf 4promoter, variant, or fragment thereof can be operably linked to any of the sequences encoding silencing elements or polypeptides disclosed herein or known in the art. In one embodiment, the Nrf4 promoter may be utilized in constructs designed to modify or alter the meiotic process. In such an embodiment, the construct may be used to express silencing elements that target TAM, OSD1, SPO11, PRD1, PRD2, PRD3, DFO1, REC8, AM1, AM2, PAM1, PAM2, AS1, DSY1, DY1, ST1, EL1, DV1, VA1, VA2 and/or PO1.

It is recognized that additional domains can be added to the promoter sequences disclosed herein and thereby modulate the level of expression, the developmental timing of expression, or the tissue type that expression occurs in. See, particularly, Australian Patent Number AU-A-77751/94 and U.S. Pat. Nos. 5,466,785 and 5,635,618.

Any of the promoter sequences employed herein can be modified to provide for a range of expression levels of the heterologous nucleotide sequence. Thus, less than the entire promoter region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of promoter deletions if, for example, there is a negative regulatory element (for a repressor) that is removed during the truncation process. Generally, at least about 20 nucleotides of an isolated promoter sequence will be used to drive expression of a nucleotide sequence.

Variant polynucleotides also encompass sequences derived from a mutagenic and recombinogenic procedure such as DNA shuffling. With such a procedure, one or more different promoter sequences can be manipulated to create a new female sporogenesis-related or ovule tissue-preferred promoter possessing the desired properties. Strategies for such DNA shuffling are described elsewhere herein.

Methods are available in the art for determining if a promoter sequence retains the ability to regulate transcription in the desired temporal and spatial pattern. Such activity can be measured by Northern blot analysis. See, for example, Sambrook et al., (1989) Molecular Cloning: A Laboratory Manual (2d ed., Cold Spring Harbor Laboratory Press, Plainview, New York), herein incorporated by reference. Alternatively, biological activity of the promoter can be measured using assays specifically designed for measuring the activity and/or level of the polypeptide being expressed from the promoter. Such assays are known in the art.

It is recognized that to increase transcription levels, enhancers may be utilized in combination with the promoter disclosed herein. Enhancers are nucleotide sequences that act to increase the expression of a promoter region. Enhancers are known in the art and include the SV40 enhancer region, the 35S enhancer element, and the like. Some enhancers are also known to alter normal promoter expression patterns, for example, by causing a promoter to be expressed constitutively when without the enhancer, the same promoter is expressed only in one specific tissue or a few specific tissues.

Modifications of the promoters disclosed herein can provide for a range of expression of the heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level or at about 1/10 transcripts to about 1/100 transcripts to about 1/1,000 transcripts.

### X. Plants and Methods of Making

The methods disclosed herein involve introducing a polypeptide or polynucleotide into a plant. “Introducing” is intended to mean presenting to the plant the polynucleotide or polypeptide in such a manner that the sequence gains access to the interior of a cell of the plant. The methods disclosed herein do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al., (1986) Biotechniques 4:320-334), electroporation (Riggs et al., (1986) Proc. Natl. Acad. Sci. USA 83:5602-5606, Agrobacterium-mediated transformation (U.S. Pat. Nos. 5,563,055 and 5,981,840), direct gene transfer (Paszkowski et al., (1984) EMBO J. 3:2717-2722), and ballistic particle acceleration (see, for example, U.S. Pat. Nos. 4,945,050; 5,879,918; 5,886,244 and 5,932,782; Tomes et al., (1995) in Plant Cell, Tissue, and Organ Culture: Fundamental Methods, ed. Gamborg and Phillips, (Springer-Verlag, Berlin); McCabe et al., (1988) Biotechnology 6:923-926), and Lec1 transformation (WO 2000/28058). Also see Weissinger et al., (1988) Ann. Rev. Genet. 22:421-477; Sanford et al., (1987) Particulate Science and Technology 5:27-37 (onion); Christou et al., (1988) Plant Physiol. 87:671-674 (soybean); McCabe et al., (1988) Bio/Technology 6:923-926 (soybean); Finer and McMullen, (1991) In Vitro Cell Dev. Biol. 27P: 175-182 (soybean); Singh et al., (1998) Theor. Appl. Genet. 96:319-324 (soybean); Datta et al., (1990) Biotechnology 8:736-740 (rice); Klein et al., (1988) Proc. Natl. Acad. Sci. USA 85:4305-4309 (maize); Klein et al., (1988) Biotechnology 6:559-563 (maize); U.S. Pat. Nos. 5,240,855; 5,322,783 and 5,324,646; Klein et al., (1988) Plant Physiol. 91:440-444 (maize); Fromm et al., (1990) Biotechnology 8:833-839 (maize); Hooykaas-Van Slogteren et al., (1984) Nature 311:763-764; U.S. Pat. No. 5,736,369 (cereals); Bytebier et al., (1987) Proc. Natl. Acad. Sci. USA 84:5345-5349 (Liliaceae); De Wet et al., (1985) in The Experimental Manipulation of Ovule Tissues, ed. Chapman, et al., (Longman, New York), pp. 197-209 (pollen); Kaeppler et al., (1990) Plant Cell Reports 9:415-418 and Kaeppler et al., (1992) Theor. Appl. Genet. 84:560-566 (whisker-mediated transformation); D'Halluin et al., (1992) Plant Cell 4:1495-1505 (electroporation); Li et al., (1993) Plant Cell Reports 12:250-255 and Christou and Ford, (1995) Annals of Botany 75:407-413 (rice); Osjoda et al., (1996) Nature Biotechnology 14:745-750 (maize via Agrobacterium tumefaciens), all of which are herein incorporated by reference.

In specific embodiments, the various sequences employed in the methods and compositions disclosed herein (e.g., the silencing elements or constructs, Nrf4 promoters or variants and fragments thereof) can be provided to a plant using a variety of transient transformation methods. Such transient transformation methods include, but are not limited to, the introduction of the various sequences employed in the methods and compositions disclosed herein (e.g., the silencing elements or constructs, Nrf4 promoters or variants and fragments thereof) directly into the plant or the introduction of the transcript into the plant. Such methods include, for example, microinjection or particle bombardment. See, for example, Crossway et al., (1986) Mol Gen. Genet. 202:179-185; Nomura et al., (1986) Plant Sci. 44:53-58; Hepler et al., (1994) Proc. Natl. Acad. Sci. 91:2176-2180, and Hush et al., (1994) Journal of Cell Science 107:775-784, all of which are herein incorporated by reference.

Alternatively, the various sequences employed in the methods and compositions disclosed herein (e.g., the silencing elements or constructs, Nrf4 promoters or variants and fragments thereof) can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethyleneimine (PEI; Sigma #P3143).

In other embodiments, the polynucleotide of the disclosure may be introduced into plants by contacting plants with a virus or viral nucleic acids. Generally, such methods involve incorporating a nucleotide construct of the disclosure within a viral DNA or RNA molecule. It is recognized that the various sequences employed in the methods and compositions disclosed herein (e.g., the silencing elements or constructs, Nrf4 promoters or variants and fragments thereof) may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta et al., (1996) Molecular Biotechnology 5:209-221, herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855, and WO 1999/25853, all of which are herein incorporated by reference. Briefly, the polynucleotide of the disclosure can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

Additional methods for targeted mutagenesis in vivo are known. For example, a DNA sequence having the desired sequence alteration can be flanked by sequences homologous to the genomic target. One can then select or screen for a successful homologous recombination event. See U.S. Pat. No. 5,527,695. Generally, such a vector construct is designed having two regions of homology to the genomic target which flank a polynucleotide having the desired sequence. Introduction of the vector into a plant cell will allow homologous recombination to occur and to produce an exchange of sequences between the homologous regions at the target site.

Such methods of homologous recombination can further be combined with agents that induce site-specific genomic double-stranded breaks in plant cells. Such double strand break agents can be engineered to produce the break at a targeted site and thereby enhance the homologous recombination events. See, for example, Puchta et al., (1996) Proc Natl Acad Sci USA 93:5055-5060; US Patent Application Publication Number 2005/0172365A1; US Patent Application Publication Number 2006/0282914, WO 2005/028942; WO 2004/067736 published Aug. 12, 2004; U.S. Pat. Nos. 5,792,632; 6,610,545; Chevalier et al., (2002) Mol Cell 10:895-905; Chevalier et al., (2001) Nucleic Acids Res 29:3757-3774; Seligman et al., (2002) Nucleic Acids Res 30:3870-3879; US Patent Application Publication Number 2009/0133152, and WO 2005/049842, each of which is herein incorporated by reference in their entirety.

The cells that have been transformed may be grown into plants in accordance with conventional ways. See, for example, McCormick et al., (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure, for example, an expression cassette of the disclosure, stably incorporated into their genome.

As used herein, the term plant includes plant cells, plant protoplasts, plant cell tissue cultures from which plants can be regenerated, plant calli, plant clumps, and plant cells that are intact in plants or parts of plants, such as embryos, pollen, ovules, seeds, leaves, flowers, branches, fruits, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The methods and compositions disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), Sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis, such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present disclosure include, for example, pines such as loblolly pine (Pinus taeda), slash pine (Pinus elliotii), ponderosa pine (Pinus ponderosa), lodgepole pine (Pinus contorta), Monterey pine (Pinus radiata), Douglas fir (Pseudotsuga menziesii), Western hemlock (Tsuga canadensis), Sitka spruce (Picea glauca), redwood (Sequoia sempervirens), true firs, such as silver fir (Abies amabilis) and balsam fir (Abies balsamea), and cedars, such as Western red cedar (Thuja plicata) and Alaska yellow cedar (Chamaecyparis nootkatensis). In specific embodiments, plants of the present disclosure are crop plants (for example, corn, alfalfa, sunflower, Brassica sp., soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica sp., maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

### XI. Methods of Use

Also described are methods for producing viable non-reduced, or non-reduced and non-recombined, gametes as well as and methods of maintaining heterozygosity in offspring. In some embodiments, the method comprises introducing into a plant a composition comprising a silencing element that reduces the level of a Nrf4 target polynucleotide, thereby producing viable non-reduced, or non-reduced and non-recombined, gametes. In other embodiments, the method comprises modifying endogenous Nrf4 target polynucleotides, for example, Nrf4 genes, using gene editing technologies, such as endonucleases, megenucleases, CRISPR-Cas guideRNA's or other polynucleotide guided double strand break reagentz or combinations thereof, so that the level or activity of a Nrf4 target polynucleotide or Nrf4 polypeptide are reduced, thereby producing viable non-reduced, or non-reduced and non-recombined, gametes.

As described elsewhere herein, the silencing element can be introduced in a variety of ways. The silencing element can be expressed in a specific manner, for example, using inducible or tissue-preferred or developmentally regulated promoters that are discussed elsewhere herein. In one embodiment, the silencing element is operably linked to a Nrf4 promoter, variant or fragment thereof. In specific embodiments, the silencing element is expressed in a plant ovule primordia, plant ovule, plant female sporocyte, plant female gametophyte, or plant female gamete.

In certain embodiments, the methods and compositions include a plant cell that has the modified endogenous Nrf4 gene or silencing element targeting Nrf4. The plant may produce non-reduced, or non-reduced and non-recombined, female gametes. Additionally, the modified Nrf4 gene or Nrf4 silencing element can be combined with other genes that are modified and/or other silencing elements that target other genes of interest.

For example, the plant cell having the modified endogenous Nrf4 gene or silencing element targeting Nrf4 may be combined or stacked with a silencing element that targets genes that play a role in recombination in order to create plants that produce, or non-reduced and, non-recombined, female gametes. For example, the recombination target genes include but are not limited to SPO11, PRD1 (De Must et al., (2007) EMBO J. 26:4126-4137), OSD1, TAM, REC8 (known as Fad in maize) Ago 104 (Singh et al., (2011) Plant Cell. 23:443-458), AM1, AM2, PAM1, PAM2, AS1, DSY1, DY1, ST1, EL1, DV1, VA1, VA2, PO1, and the like and combinations thereof.

In other embodiments, the plant cell having the modified endogenous Nrf4 gene or silencing element targeting Nrf4 may be combined with one or more genes involved in recombination that have been modified, for example, by gene-editing technologies, to have decreased recombination activity in order to create plants that produce non-reduced, or non-reduced and non-recombined, female gametes. The non-reduced, or non-reduced and non-recombined, female gametes therefore may be produced using gene-editing techniques or silencing elements or combinations thereof.

The non-reduced, or non-reduced and non-recombined, female gametes can be stacked with traits desirable for disease or herbicide resistance (e.g., fumonisin detoxification genes (U.S. Pat. No. 5,792,931); avirulence and disease resistance genes (Jones et al., (1994) Science 266:789-793; Martin et al., (1993) Science 262:1432-1436; Mindrinos et al., (1994) Cell 78:1089-1099); acetolactate synthase (ALS) mutants that lead to herbicide resistance, such as the S4 and/or Hra mutations; inhibitors of glutamine synthase, such as phosphinothricin or basta (e.g., bar gene); and glyphosate resistance (EPSPS gene); traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al., (1988) J. Bacteriol. 170:5837-5847) facilitate expression of polyhydroxyalkanoates (PHAs)), the disclosures of which are herein incorporated by reference. One could also combine various polynucleotides, for example, polynucleotides providing agronomic traits such as male sterility (e.g., see U.S. Pat. No. 5,583,210), stalk strength, flowering time, or transformation technology traits, such as cell cycle regulation or gene targeting (e.g., WO 99/61619, WO 00/17364, and WO 99/25821), the disclosures of which are herein incorporated by reference.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

A further embodiment includes methods of maintaining heterozygosity in a progeny plant that includes regenerating a progeny plant from a parent plant that has non-reduced, non-recombinant clonal gametes and has the same genotype as the parent plant. In some examples, the endogenous Nrf4 gene and/or gene involved in recombination is disrupted in the parent and progeny plant. In an aspect, the methods disclosed herein can further comprise the step of introducing into the plant genome a disruption of the endogenous Nrf4 gene, and regenerating a plant having such an altered genome.

In one example, compositions include an isolated polynucleotide comprising a nucleotide sequence comprising at least 19 consecutive nucleotides of any one of SEQ ID NOS: 3, 4, 5, 6, 7, 8, 10, 12, 13, 17, 18, 22, 28 and 29 or a complement thereof, wherein said polynucleotide encodes a silencing element that decreases Nrf4 activity. In other embodiments, compositions include an isolated polynucleotide comprising a nucleotide sequence comprising nucleotide sequence that hybridizes under stringent conditions to the full length complement of the nucleotide sequence of comprising any one of SEQ ID NOS: 3, 4, 5, 6, 7, 8, 10, 12, 13, 17, 18, 22, 28 and 29, wherein said polynucleotide encodes a silencing element that decreases Nrf4 activity. In some embodiments, the silencing element is part of an expression cassette operably linked to a female sporegenesis-related promoter. An expression cassette may express a polynucleotide disclosed herein as a double stranded RNA. An expression cassette comprising a heterologous polynucleotide disclosed herein, wherein said polynucleotide comprises a silencing element, which is expressed as a hairpin RNA.

### XII. Methods of Modulating Nrf4 Activity and/or Concentration

Methods and compositions are provided to modify the expression level and/or activity of a Nrf4 polynucleotide and/or Nrf4 polypeptide in a plant cell, for example, an ovule primordia, ovule, female sporocyte, female gametophyte, or female gamete plant cell. In some embodiments, methods and compositions include decreasing the Nrf4 activity or expression level of a Nrf4 polynucleotide, Nrf4 polypeptide, variant or fragment thereof in a plant cell, for example, an ovule primordia, ovule, female sporocyte, female gametophyte, or female gamete. Compositions suitable for this purpose are described elsewhere herein.

In some embodiments, methods and compositions include increasing the Nrf4 activity or expression level of a Nrf4 polynucleotide, Nrf4 polypeptide, variant, orthologs or fragment thereof in a plant cell, for example, in an ovule primordia, ovule, female sporocyte, female gametophyte, or female gamete.

Biologically active variants of a Nrf4 polynucleotide will have at least about 70%. 75%, 80%, 85%, 86%, 87%, 88%, 89%, 90%, 91%, 92%, 93%, 94%, 95%, 96%, 97%, 98%, 99% or more sequence identity to any polynucleotide encoding a Nrf4 polypeptide, including the polynucleotide of any one of SEQ ID NO: 3, 4, 5, 6, 7, 8, 10, 12, 13, 17, 18, 22, 28, and 29 as determined by sequence alignment programs and parameters described elsewhere herein.

Biologically active variants of an Nrf4 polypeptide (and the polynucleotide encoding the same) will have at least about 70%. 75%, 80%, 85%, 86%, 87%, 88%, 89%, 90%, 91%, 92%, 93%, 94%, 95%, 96%, 97%, 98%, 99% or more sequence identity to any Nrf4 polypeptide, including but not limited to, the polypeptide of any one identified in Table 1, for example, as set forth in SEQ ID NOs: 1, 2, 9, 14, 16, 20, 24, 25, or 27 as determined by sequence alignment programs and parameters described elsewhere herein.

As discussed elsewhere herein, methods and compositions are provided which employ polynucleotides and polypeptides having Nrf4 activity. Fragments and variants of Nrf4 polynucleotides and Nrf4 polypeptides are also encompassed. By “fragment” is intended a portion of the polynucleotide or a portion of the amino acid sequence and hence protein encoded thereby. Fragments of a polynucleotide may encode protein fragments that retain non-reduction and/or non-recombination activity. Thus, fragments of a nucleotide sequence may range from at least about 20 nucleotides, about 50 nucleotides, about 100 nucleotides and up to the full-length polynucleotide encoding the Nrf4 polypeptides.

A fragment of a Nrf4 polynucleotide that encodes a biologically active portion of a Nrf4 protein will encode at least 50, 75, 100, 150, 175, 200, 225, 250, 275, 300, 325, 350, 375, 400, 410, 415, 420, 425, 430, 435 or 440 contiguous amino acids or up to the total number of amino acids present in a full-length Nrf4 polypeptide.

Thus, a fragment of a Nrf4 polynucleotide may encode a biologically active portion of a Nrf4 polypeptide. A biologically active portion of a Nrf4 polypeptide can be prepared by isolating a portion of one of the Nrf4 polynucleotides, expressing the encoded portion of the Nrf4 polypeptides (e.g., by recombinant expression in vitro), and assessing the activity of the portion of the Nrf4 protein. Polynucleotides that are fragments of a Nrf4 nucleotide sequence comprise at least 16, 20, 50, 75, 100, 150, 200, 250, 300, 350, 400, 450, 500, 550, 600, 650, 700, 800, 900, 1,000, 1,100, 1,200, 1,300 or 1,400 contiguous nucleotides or up to the number of nucleotides present in a full-length Nrf4 polynucleotide disclosed herein.

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein, that is, have non-reduction activity. Such variants may result from, for example, genetic polymorphism or from human manipulation.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the Nrf4 polypeptides. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode a Nrf4 polypeptide.

The Nrf4 polypeptides, active variants and fragments thereof may be altered in various ways including amino acid substitutions, deletions, truncations and insertions. Methods for such manipulations are generally known in the art. For example, amino acid sequence variants and fragments of the Nrf4 proteins can be prepared by mutations in the DNA. Methods for mutagenesis and polynucleotide alterations are well known in the art. See, for example, Kunkel, (1985) Proc. Natl. Acad. Sci. USA 82:488-492; Kunkel et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found. Washington, D.C.), herein incorporated by reference. Conservative substitutions, such as exchanging one amino acid with another having similar properties, may be optimal.

Thus, the genes and polynucleotides disclosed herein include both the naturally occurring sequences as well as DNA sequence variants. Likewise, the Nrf4 polypeptides and proteins encompass bothnaturally occurring polypeptides as well as variants and modified forms thereof. Mutations that will be made in the DNA encoding the variant must not place the sequence out of reading frame and optimally will not create complementary regions that could produce secondary mRNA structure. See, EP Patent Application Publication Number 75,444.

Variant polynucleotides and proteins also encompass sequences and proteins derived from a mutagenic and recombinogenic procedure such as DNA shuffling. With such a procedure, one or more different coding sequences can be manipulated to create a new Nrf4 polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the Nrf4 sequences disclosed herein and other known Nrf4 genes to obtain a new gene coding for a protein with an improved property of interest, such as a decreased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri et al., (1997) Nature Biotech. 15:436-438; Moore et al., (1997) J. Mol. Biol. 272:336-347; Zhang et al., (1997) Proc. Natl. Acad. Sci. USA 94:4504-4509; Crameri et al., (1998) Nature 391:288-291, and U.S. Pat. Nos. 5,605,793 and 5,837,458.

Thus, constructs are provided comprising a promoter operably linked to a Nrf4 polynucleotide that when expressed has Nrf4 activity in a plant female gamete, female gametophyte, female sporocyte, ovule, or ovule primodium. In one embodiment, compositions include an isolated polynucleotide comprising a nucleotide sequence comprising polynucleotides 70%, 75%, 80%, 85%, 90%, 91%, 92%, 93%, 94%, 95%, 96%, 97%, 98% or 100% identical to of any one of the polynucleotides of SEQ ID NOS: 3, 4, 5, 6, 7, 8, 10, 12, 13, 17, 18, 22, 28 and 29 or a complement thereof.

In still further embodiments, the polynucleotide encodes for a polypeptide set forth in SEQ ID NO: 1, 2, 9, 14, 16, 20, 24, 25 and 27 or an Nrf4 polynucleotide encoding a Nrf4 polypeptide having at least 80%, 85%, 90%, 91%, 92%, 93%, 94%, 95%, 96%, 97%, 98% 99% or 100% sequence identity to the polypeptide set forth in SEQ ID NO: 1, 2, 9, 14, 16, 20, 24, 25 and 27.

The following examples are offered by way of illustration and not by way of limitation.

### EXAMPLES

The embodiments are further defined in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of the embodiments, and without departing from the spirit and scope thereof, can make various changes and modifications of them to adapt to various usages and conditions. Thus, various modifications of the embodiments in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1: Genetic Screen and Identification of the Maize (Zea mays L.) Nrf4 Mutant

A genetic screen was conducted to identify mutants which form viable, non-reduced female gametes. The screen makes use of the developmental failure of maize endosperm that does not possess the normal 2 maternal to 1 paternal (2m:1p) genome ratio (Lin, (1984) Genetics 100:475-486). When wild-type diploid plants, which produce normal, reduced (haploid) female gametes, are pollinated with diploid pollen (containing diploid male gametes) from a tetraploid parent, fertilization of the central cells results in imbalanced endosperm (2m:2p), leading to seed abortion. Plants of a mutagenized M₂ population that produce ears with plump seeds, presumably with a 4m:2p genome ratio in the endosperm, may possess a mutation producing non-reduced (diploid), functional female gametes.

Maize germplasm with very active Mutator (Mu) transposable elements at a high copy number were crossed to the public inbred line Oh43, and the resulting F₁ plants were self-pollinated to create M₂ families, which should segregate 1 homozygous wild-type (no insertion): 2 insertion heterozygous: 1 insertion homozygous plant for any given insertion. Screening at the M₂ rather than F₁ stage (Singh et al., (2011) supra), allows the discovery of recessive mutants affecting female sporogenesis including meiosis (Grossniklaus, (2001) supra). Approximately 3200 M₂ families (˜15 to 20 plants each) were subsequently crossed as females with a tetraploid line. The tetraploid tester line contains the dominant embryo/endosperm color marker R1-navajo (R1-nj) to insure that seed was indeed produced by cross-pollination rather than unintended self- or sib-pollination between or within the female M₂ families. The tetraploid R1-nj line was specifically generated for this purpose by crossing a diploid R1-nj pollen parent to a public tetraploid W23 line that had been generated using the elongate1 method (Birchler, (1994) in The Maize Handbook, eds. Freeling, Walbot (Springer, Berlin), pp. 394-395). Taking advantage of some diploid pollen that is produced spontaneously, rare tetraploid offspring with a 4m:2p genome ration in the endosperm can be recovered. Subsequent self- and sib-pollinations and selection for high pollen viability led to the isolation of a fully fertile, tetraploid tester line that is tetraplex for the R1-nj allele (R1-nj/R1-nj/R1-nj/R1-nj).

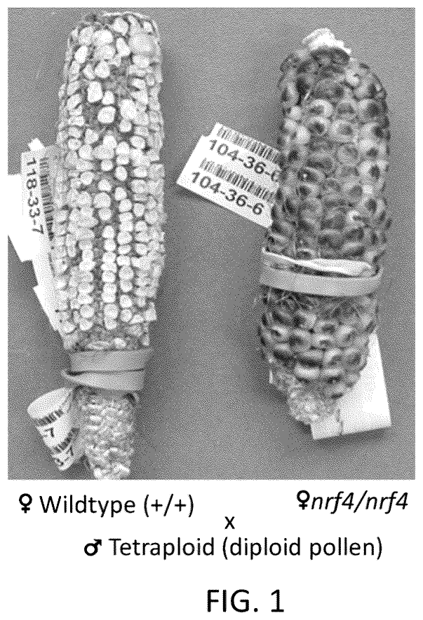

In this screen, four families were identified with plants possessing ears with plump (non-aborted) seeds. One of them, designated non-reductive in female4 (nrf4), contained a few ears with a very high proportion of plump seeds (FIG. 1). Progeny growth from a portion of such seeds confirmed viability and tetraploidy. In addition to crossing the nrf4 mutant as females with the tetraploid line, the M₂ plants were used as males in crosses with the public inbred line W22. Individual plants that produced a high frequency of plump seeds also produced a high frequency of plump seeds in the cross with W22 (diploid), indicating that non-reduction was female-specific. The F₁ plants produced from the cross of W22×high-frequency plump-seeded plants were self-pollinated, and the resulting F₂ plants were crossed as females with tetraploid plants to confirm the initial phenotype. Out of a total of 57 ears evaluated, 14 produced a high frequency of plump seeds up to 85-100%; this fits a model of a monogenic recessive trait (χ², 1 d.f.=0.94). This frequency (penetrance) of non-reduced, viable female gametes per ear is generally higher than reported for other maize mutants (30-60%, elongate1 (el1; Rhoades and Dempsey, (1966) Genetics 54:505-22); 20-80%, Dominant non-reduction 4 (Dnr4; Singh et al., 2011), supra).

### Example 2: Cloning of the Nrf4 Allele

Plants producing plump seed in the second evaluation in Example 1 were crossed again as males to the public inbred line W22, the new F₁ was self-pollinated, and the resulting F₂ plants were once again crossed as females with the tetraploid R1-nj tester line. Samples of leaf tissue were taken from each pollinated plant. Fifteen plants which were subsequently shown to produce a high percentage of plump seed were evaluated by high-throughput sequencing (HTS) analysis of their Mu-flanking regions. This method utilizes the sequence in the terminal-inverted-repeat (TIR) region, which is conserved in all Mu elements.

DNA was isolated from the 15 samples described above. DNA was normalized to 20 ng/ul, sheared on Covaris E210 (10% Duty Cycle, Intensity of 0.5, 1000cycles/burst for 45 seconds), and processed through the Flanking Sequence library prep method. This library preparation involved repairing the DNA ends using the NEBNext End Repair Enzyme mix; followed by adding an A-base to the 3′ end of the DNA with NEBNext Klenow Fragment. An indexed adapter was then ligated using NEBNext Quick T4 DNA ligase (New England Biolabs). Libraries undergo 2 rounds of PCR (PCR1, PCR2) using 2× Phusion DNA Polymerase Mastermix (New England Biolabs), for 18 and 20 rounds of amplification, respectively. The 1^{st} primer in PCR1 (PHN130859-Muint26) and PCR2 (PHN130859-Muint26-PE2.0) has a sequence that matches the common region in all Mutator elements, except that PCR2 primers had specific sequences attached to enable Illumina sequencing. The 2^{nd} primer in each reaction (PE1.0) has sequence that matches the ligated adapter. For nucleotide sequences of all primers see Table 2.

TABLE 2

| Mu Oligo | |
|---|---|
| name | Sequence |
| PHN130859- | AGAAGCCAACGCCAWCGCCTCYATTTC |
| Muint26 | (SEQ ID NO: 30) |
| PHN130859- | CAAGCAGAAGACGGCATACGAGATAGAAGCCAAC |
| Muint26-PE2.0 | GCCAWCGCCTCYATTTC (SEQ ID NO: 31) |
| PE1.0 | AATGATACGGCGACCACCGAGATCTACACTCTTT |
| | CCCTACACGACGCTCTTCCGATCT |
| | (SEQ ID NO: 32) |

After PCR amplification, the products were purified using Ampure XP beads and ran on a Fragment Analyzer (Advanced Analytical Technologies Inc.) to check for quality and quantity. Products of all samples were pooled and sequenced on an Illumina HiSeq2000 or GAIIx sequencer with a run configuration of single read 108 bases. Bases 1 to 6 are the barcode to identify the sample, base 7 is a T-base and is removed. This leaves 101 bp of sequence for analysis. nrf4 events had approximately 1.5-10 million reads for each sample.

One Mu-flanking sequence was found to be present in all 15 plants, and thus most likely to be the causal insertion of the nrf4 non-reduction phenotype. Reverse genetics based on TUSC (Meeley and Briggs, (1995) Maize Genetics Cooperation Newsletter 69:67-82) was used to isolate two additional Mu insertion alleles in the same gene, and they also showed the non-reductive phenotype. Thus, the correct identity of the isolated Nrf4 gene was clearly confirmed using additional alleles. The Mu insertions are located in locus GRMZM2G148133 (on the world wide web at http://www.maizegdb.org/cgi-bin/displaygmresults.cgi?term=GRMZM2G148133), which has homologs in several grass species but does not have any functional annotation (SEQ ID NOs #17-19).

### Example 3: MicroRNA Experiment to Phenocopy the Nrf4 Mutant

Based on the Nrf4 gene sequence, a micro RNA sequence was designed against the Nrf4 coding region (see SEQ ID NOs #33-37). This sequence was inserted into a transformation vector using the maize ubiquitin promoter to drive expression. Transformed maize plants carrying a single copy of the construct were generated. These plants were used as females in crosses with diploid male plants, which produced normal haploid pollen. As a result of these crosses the transformed plants showed seed set, indicating that they had reduced female gametes as well, and that the nrf4 non-reduced female gamete phenotype was not achieved with this particular microRNA design. A proprietary software program for gene silencing indicated the microRNA sequence had folding at its 5′ end, and without wishing to be bound by this theory, it is possible that this design affected microRNA function.

### Example 4: Expression Analysis of Nrf4 Transcript Using in Situ Hybridization

Based on the Nrf4 gene sequence, an antisense gene-specific probe was designed covering the first, second, and part of the third exon of the Nrf4 gene. This Nrf4 probe sequence was inserted into the pDRIVE vector (Qiagen) suited for in vitro transcription. A sense riboprobes labeled with digoxygenin (DIG) was synthesized using the SP6 polymerase.

Immature wild-type ears of inbred line W22 (approximately 3-4 cm in size) were fixed, embedded in paraffin, and sectioned using a microtome. Tissue sections were subjected to RNA in situ hybridization with the riboprobe described above. The riboprobe was detected using anti-DIG-antibodies fused with alkaline phosphatase (Roche) and the signal was developed by providing the alkaline phosphatase substrate Western Blue (Roche). Images were taken on a transmission light microscope equipped with a digital color camera (Leica).

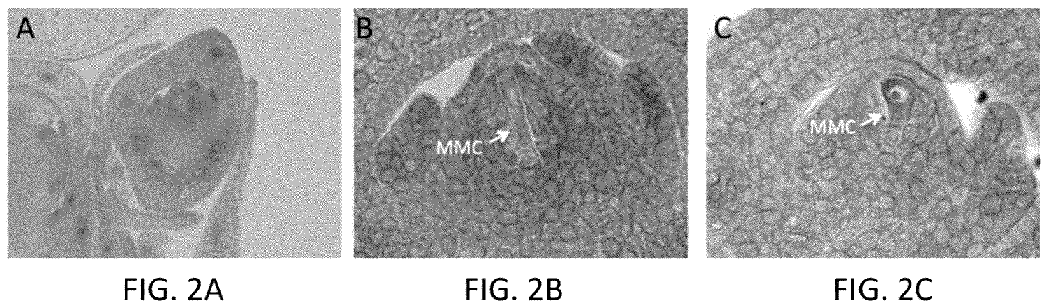

The Nrf4 transcript showed a consistently high expression in a portion of the ovule's nucellus around megaspore mother cell (MMC) and in the integument primordia (FIG. 2A). In the developing and mature MMC, the Nrf4 signal varied from intermediate to very strong depending on the particular section (FIG. 2B & FIG. 2C). This variation may related to different stages of MMC differentiation that are cytologically not distinguishable in these sections.

### Example 5: Expression Analysis of the Nrf4 Gene

A DNA sequence primarily consisting of the putative promoter region of the Nrf4 gene (nt 1-1737 of SEQ ID NO: 7) was synthesized (GenScript), with the following modifications: 32bp were added 5′ in order to add an EcoRI restriction site, and STOP codons in all 6 reading frames; nt 521 was changed from G to C to eliminate an Eco RI restriction site; nt 841 was changed from A to T to eliminate an ORF; and nt 1737 was changed from A to G to be compatible with a BamHI restriction site. The resulting DNA was cloned into a vector cut with EcoRI and BamHI, creating a plant transcriptional unit (PTU) consisting of the putative Nrf4 promoter, Adh1 intron, the ZS-green1 fluorescent protein (Clontech) CDS, and the pinII transcription terminator. This entry vector was subsequently cloned into a plant transformation vector using Gateway multisite cloning (Invitrogen), introduced into Agrobacterium tumefaciens by electroporation, and transformed into maize by standard methods. Such methods for preparing the constructs and transforming maize are as previously described and known in the art.

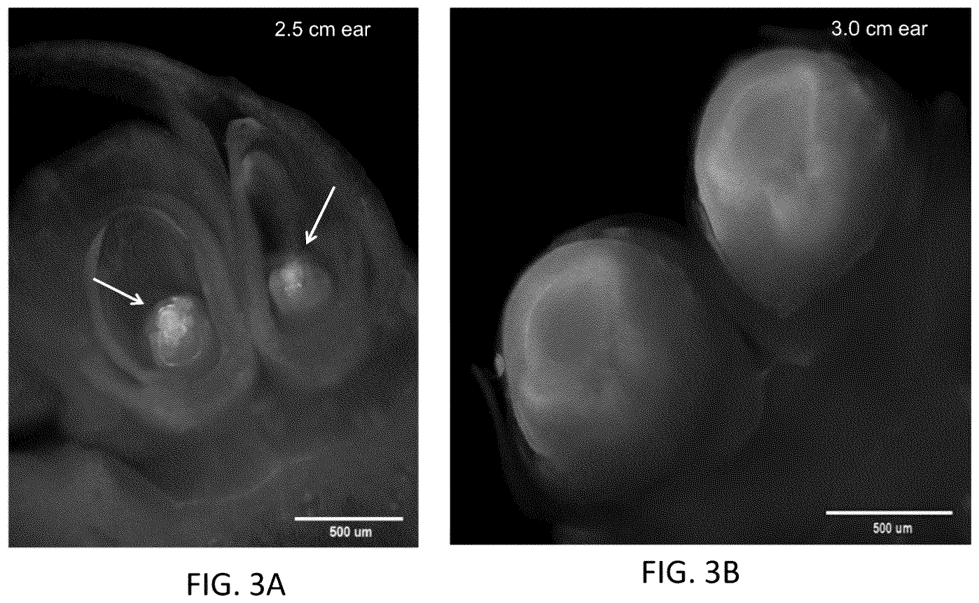

Immature ears (approximately 2.5 and 3 cm in size) of regenerated transgenic plants were examined. Hand cross-sections were made through the ear, but horizontal longitudinal through the axis of the ovule primordia. Images were observed on an epi-fluorescence microscope using a GFP filter set for the Zs-green1 and a DAPI filter set for the natural blue auto-fluorescence of the tissue. Ovule-specific Zs-green1-positive fluorescence was observed in the 2.5 cm ear, which is pre-meiotic in development, prior to female sporocyte differentiation (FIG. 3A). Similar expression was also observed in the 3 cm ear sections, at which stage female sporocytes are fully differentiation, but also pre-meiotic (FIG. 3B). Consequently, the Nrf4 promoter may be utilized in constructs designed to modify or alter the meiotic process, a key step in apomixis.

### Example 6: Production of a Tetraploid “Haploid” Inducer

Marker analysis of female meiotic behavior in non-reduced eggs can be simplified by elimination of the male genome. In maize, some lines (generally referred to as “haploid inducers”) produce pollen capable of inducing gynogenesis, resulting in the production of maternal haploid progeny that originate exclusively from the egg cell (Röber et al., (2005) Maydica 50:275-283). A major locus, designated gynogenesis inducer1 (ggi1), controls gynogenesis in maize (Barret et al., (2008) Theor. Appl. Genet. 117:581-594).

Haploid inducers may possess a dominant marker gene to distinguish biparental diploid from maternal haploid progeny. One example is R1-nj, which produces a purple scutellum and a purple crown of the aleurone (endosperm) of F₁ kernels crossed with unpigmented females. In haploid induction crosses, kernels with a maternally-derived haploid embryo and a regular triploid endosperm possess a colorless embryo (scutellum) and a purple crown (endosperm), whilst F₁ (diploid) kernels have pigmentation in both embryo and endosperm.

In order to eliminate the male genome in mutants with non-reduced egg (and central) cells and produce viable seeds, an inducer must be created which is tetraploid (to preserve the 2m-1p genome ratio in the endosperm). To accomplish this, the fact that normal diploid maize will produce a certain low frequency of diploid eggs (0.5-5/1000; Bauman, (1961) Maize Genet. Coop. Newslet. 35:128-130) was exploited. Consequently, the R1-nj tetraploid maize line described above was used as a male in a cross with a diploid inducer line (R1-nj, ggi1), and the rare plump kernels were selected. Seeds from these kernels were grown and confirmed to be tetraploid by standard flow cytometry. Such plants would be homozygous for R1-nj and heterozygous for ggi1; specifically as a duplex tetraploid (ggi1/ggi1/Ggi1/Ggi1). These plants were self-pollinated. The progeny were analyzed for markers flanking ggi1, and homozygous ggi1 plants (frequency 1/36) were selected. These plants were also self-pollinated and crossed as a male to a tetraploid line lacking R1-nj. Approximately 5% of the progeny possessed a colorless embryo and a purple crown. Flow cytometry confirmed that these progeny were in fact diploid. This tetraploid version of a “haploid” inducer (THI) was used in crosses with nrf4 homozygous females to generate maternal diploid offspring.

### Example 7: Marker Analysis of Nrf4 Meiotic Behavior Demonstrates the Production of Maternal Clones

In order to test for possible background/modifier effects on the nrf4 phenotype and to insure a high frequency of heterozygous markers for analysis, the nrf4 mutant allele was crossed 4 times (BC₃) into 4 different inbred lines (Public lines B73 & W22; and 2 Pioneer proprietary lines Line Z & Line Y). The converted inbred lines were crossed to each other, and nrf4 homozygotes were identified. The F₁ nrf4 homozygous plants were crossed as females with THI (described in Example 5). Diploid progeny of maternal origin was selected as described in Example 5, with the additional step of confirming the lack of ggi1-flanking markers. The key to self-reproducing hybrids (SRH), as in naturally occurring apomicts, is progeny that are identical to the maternal parent. In addition to non-reduction, this requires a lack of recombination and chromosome segregation, resulting in the full retention of heterozygosity. To assess the level at which heterozygosity is retained in the progeny, both the female F₁ nrf4 homozygous maternal plants and their selected diploid progeny were tissue-sampled. Both DNA preparation and single-nucleotide polymorphism (SNP) analysis were conducted using standard methods known to one of ordinary skill in the art. A total of 384 SNP markers, distributed as evenly as possible across the maize genome, were analyzed. Depending on the particular F₁, and the particular plant within a cross, the number of informative (heterozygous in the maternal plant) markers varied (Table 3). In total, 10 out of 227 progeny (4.4%) retained 100% of the heterozygosity present in its maternal parent. Thus, nrf4 is capable of producing “clonal” gametes, albeit at a low frequency. The differences in the frequency within the individual F₁ hybrids may indicate the presence of background effects or modifier gene(s), which may be exploited through selection to produce higher levels of clonal female gametes.

TABLE 3

| | Number | | Number of |
|---|---|---|---|
| | of Parent- | Number | Progeny Retaining |
| | Progeny | of Informative | 100% |
| F₁ Pedigree | Pairs | Markers Per Pair | Heterozygousity |
| B73 × Line Z | 63 | 117-181 | 4 (6.3%) |
| Line Y × Line Z | 18 | 100-109 | 2 (11%) |
| W22 × B73 | 111 | 105-197 | 5 (4.5%) |
| W22 × Line Z | 28 | 122-143 | 0 (0%) |
| W22 × Line Y | 7 | 140 | 0 (0%) |

### Example 8: Combining Nrf4 With Other Mutants

Depending on the exact mechanisms of non-reduced gamete production, their genetic content may differ substantially (Crismani et al., (2013) J. Exp. Bot. 64:55-65). Two aspects affect the genotype of 2n gametes: (1) segregation of homologous or sister chromatids (centromeres), and (2) the presence or absence of recombination (Crismani et al., (2013) supra). In some cases, the genetic products of meiosis show that sister centromeres segregate from each other, but recombination occurs. Consequently, heterozygosity is preserved at centromeres and tends to be reduced away from the centromeres. This is generally referred to as a first division restitution (FDR) mechanism. Examples include ps1 and jason mutants in Arabidopsis (Crismani et al., (2013) supra). In other cases, the opposite effect occurs as sister chromatids (centromeres) migrate together (recombination occurs). Heterozygosity is completely lost at the centromeres, and the resulting homozygosity tends to be reduced with distance from the centromeres. This is sometimes referred to as second division restitution (SDR). Examples include osd1 and tam in Arabidopsis (Crismani et al., (2013) supra). Thus, beyond non-reduced gamete production, the engineering of apomixis in a species requires a mitotic-like division to replace meiosis-sister chromatids segregating without recombination. This has been accomplished in Arabidopsis at a high frequency in the triple mutant spo11 rec8 osd1 (MiMe; d'Erfurth et al., (2009) PLoS Biology 7: e1000124), although the level of female non-reduced spores in osd1 mutants is estimated to be ˜85%.

Despite the low frequency of occurrence, the ability of nrf4 mutants to produce non-reduced, non-recombined but viable gametes makes Nrf4 a good candidate for the engineering of apomixis. Indeed, maternal clonal progeny could be produced in crosses with THI. Given its overall very high degree of non-reduced female gamete viability, nrf4 may be combined with other mutants in order to increase the frequency of non-recombination and replace meiosis with a mitotic-like division. In particular, mutants which completely eliminate recombination, such as spo11 and prd1 (De Muyt et al., (2007) EMBO J. 26:4126-4137). Also, nrf4 could serve as a substitute for osd1 or tam in combination with rec8 (known as afd1 in maize) and spo11 in a maize version analogous to Arabidopsis MiMe. Other known maize mutants affecting meiosis, which could be combined with nrf4, are ago104 (Singh et al., (2011) supra) as well as am1, am2, pam1, pam2, as1, dsy1, dy1, st1, el1, dv1, va1, va2, and po1 (Table 12: Meiotic Mutants, (1997) in Mutants of Maize, eds. Neuffer, Coe, Wessler (Cold Spring Harbor Laboratory Press, New York), p. 314).

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

We claim:

1. A method of producing viable non-reduced, or viable non-reduced and non-recombined, gametes, the method comprising: disrupting an endogenous Nrf4 gene in a plant or plant cell, wherein the plant cell is an ovule primordia, ovule, female gametophyte, or female gamete, thereby producing viable non-reduced, or viable non-reduced and non-recombined, gametes.

2. The method of claim 1, wherein the endogenous Nrf4 gene comprises a polynucleotide selected from the group consisting of:
a) a polynucleotide having at least 70% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
b) a polynucleotide having at least 80% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
c) a polynucleotide having at least 90% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
d) a polynucleotide selected from the group consisting of SEQ ID NOs: 3, 4, 5, 6, 7, 8, 10, 12, 13, 17, 18, 22, 28, and 29;
e) a polynucleotide that is a variant of the polynucleotide of (a), (b), (c) or (d);
f) a polynucleotide that is a fragment of the polynucleotide of (a), (b), (c), (d) or (e);
g) a polynucleotide that is complementary to the polynucleotide of (a), (b), (c), (d), (e) or (f); and
h) a polynucleotide that hybridizes under stringent conditions to the polynucleotide of (a), (b), (c), (d), (e), (f) or (g).

3. The method of claim 1, wherein the endogenous Nrf4 gene encodes for a Nrf4 polypeptide selected from the group consisting of:
a) a polypeptide comprising an amino acid sequence being identical to or having at least 70% identity with SEQ ID NOs: 1, 2, 9, 14, 16, 20, 24, 25 or 27, or an ortholog thereof;
b) a polypeptide comprising an amino acid sequence being identical to or having at least 80% identity with SEQ ID NOs: 1, 2, 9, 14, 16, 20, 24, 25 or 27, or an ortholog thereof;
c) a polypeptide comprising an amino acid sequence being identical to or having at least 90% identity with SEQ ID NOs: 1, 2, 9, 14, 16, 20, 24, 25 or 27, or an ortholog thereof;
d) a polypeptide comprising an amino acid sequence being identical to or having at least 100% identity with SEQ ID NOs: 1, 2, 9, 14, 16, 20, 24, 25 or 27, or an ortholog thereof;
e) a polypeptide comprising an amino acid sequence, which is a variant of the amino acid sequence of (a), (b), (c) or (d); and
f) a polypeptide comprising an amino acid sequence which is a fragment of the amino acid sequence of (a), (b), (c), (d), or (e).

4. The method of claim 1, wherein said disruption inhibits expression, activity, or both expression and activity of a product of said endogenous Nrf4 gene compared to a corresponding control plant lacking such a disruption.

5. The method of claim 1, further comprising a step of disrupting in the plant, in addition to the Nrf4 gene, at least one other endogenous gene involved in meiosis.

6. The method of claim 1, further comprising a step of disrupting in the plant, in addition to the Nrf4 gene, at least one other gene involved in recombination.

7. The method of claim 5, wherein the at least one other endogenous gene involved in meiosis is REC8 or an ortholog thereof.

8. The method of claim 7, further comprising disrupting one of the following genes selected from the group consisting of SPO11, PRD1, PRD2, PRD3, DFO1, and any orthologs thereof.

9. The method of claim 7, further comprising disrupting OSD1 or TAM1, or orthologs thereof, or any combination thereof.

10. The method of any of the claims 1-9, wherein the female gametophyte or female gamete undergoes parthenogenesis or genome elimination.

11. The method of claim 10, further comprising producing a seed, wherein the seed comprises parthenogenetically-derived clonal embryos.

12. The method of claim 1, further comprising regenerating a plant having the disruption of the endogenous Nrf4 gene.

13. The method of claim 1, wherein the plant is maize, wheat, rice, sorghum, barley, oat, lawn grass, rye, soybean, Brassica, or sunflower.

14. The method of claim 1, wherein the plant with the disrupted endogenous Nrf4 gene comprises viable non-reduced, viable or non-reduced and non-recombined, gametes when compared to a control plant.

15. The method of any of claims 1-14, wherein the method produces a non-naturally occurring plant with developing ovules comprising non-reduced, or non-reduced and non-recombined clonal female or male gametes.

16. A non-naturally occurring plant produced by the method of any of claims 1-15, wherein the plant comprises a disruption in an endogenous Nrf4 gene and wherein the developing ovules comprise non-reduced, or non-reduced and non-recombined clonal gametes.

17. A seed of the non-naturally occurring plant of claim 16.

18. A nucleic acid construct comprising a nucleotide sequence encoding an element that silences Nrf4 activity.

19. The nucleic acid construct of claim 18, wherein the silenced Nrf4 activity is a function of reducing female sporocytes, female gametophytes, or female gametes during meiosis.

20. The nucleic acid construct of claim 18, wherein the element comprises at least 19 nucleotides of any one of SEQ ID NOs: 3, 4, 5, 6, 7, 8, 10, 12, 13, 17, 18, 22, 28, and 29 or a fragment or variant thereof, or a complement thereof.

21. The nucleic acid construct of claim 18, wherein the element comprises a nucleotide sequence that hybridizes under stringent conditions to a full length complement of a nucleotide sequence of any one of SEQ ID NOs: 3, 4, 5, 6, 7, 8, 10, 12, 13, 17, 18, 22, 28, and 29 or a fragment thereof.

22. The nucleic acid construct of claim 18, wherein the construct further comprises a promoter functional in plants operably linked to the element.

23. The nucleic acid construct of claim 22, wherein the promoter is a heterologous promoter.

24. The nucleic acid construct of claim 22, wherein said promoter is a constitutive, inducible, cell- or tissue-preferred, or cell- or tissue-specific promoter.

25. The nucleic acid construct of claim 18, wherein the element is operably linked to a promoter in sense orientation or in antisense orientation.

26. A plant cell transformed with the construct of claim 18.

27. The plant of claim 26, wherein the plant comprises viable non-reduced, or non-reduced and non-recombined, gametes compared to a control plant.

28. The plant of claim 26, wherein the plant is Arabidopsis, maize, wheat, rice, sorghum, barley, oat, lawn grass, rye, soybean, Brassica, sunflower, millet, sugarcane, cotton, safflower, tobacco, or alfalfa.

29. The seed from the plant of any one of claim 27 or 28.

30. A method of decreasing Nrf4 activity in a plant, the method comprising decreasing expression of one or more Nrf4 genes in the plant.

31. The method of claim 30, wherein the decreased expression of the one or more Nrf4 genes is in an ovule primordia, ovule, female sporocyte, female gametophyte, or female gamete of the plant.

32. The method of claim 30, further comprising introducing into the plant the nucleic acid construct of any one of claims 18-25.

33. The method of claim 32, wherein the one or more Nrf4 genes is endogenous to the plant.

34. The method of claim 32, wherein the Nrf4 gene comprises a polynucleotide selected from the group consisting of:
a) a polynucleotide having at least 70% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
b) a polynucleotide having at least 80% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
c) a polynucleotide having at least 90% sequence identity, as determined by the GAP algorithm under default parameters, to the full length sequence of a polynucleotide selected from the group consisting of SEQ ID NOs: 3, 4, 5, 6, 7, 8, 10, 12, 13, 17, 18, 22, 28, and 29, and wherein the polynucleotide encodes a polypeptide that has the function of reducing, or reducing and recombining, female sporocytes, female gametophytes, or female gametes during meiosis;
d) a polynucleotide selected from the group consisting of SEQ ID NOs: 3, 4, 5, 6, 7, 8, 10, 12, 13, 17, 18, 22, 28, and 29;
e) a polynucleotide which is a variant of the polynucleotide of (a), (b), (c) or (d);
f) a polynucleotide which is a fragment of the polynucleotide of (a), (b), (c), (d) or (e);
g) a polynucleotide which is complementary to the polynucleotide of (a), (b), (c), (d), (e) or (f); and
h) a polynucleotide that hybridizes to the polynucleotide of (a), (b), (c), (d), (e), (f) and (g).

35. The method of claim 32, wherein the endogenous Nrf4 gene encodes a polypeptide selected from the group consisting of:
a) a polypeptide comprising an amino acid sequence being identical to or having at least 70% identity with SEQ ID NOs: 1, 2, 9, 14, 16, 20, 24, 25 and 27, or an ortholog thereof;
b) a polypeptide comprising an amino acid sequence being identical to or having at least 80% identity with SEQ ID NOs: 1, 2, 9, 14, 16, 20, 24, 25 and 27, or an ortholog thereof;
c) a polypeptide comprising an amino acid sequence being identical to or having at least 90% identity with SEQ ID NOs: 1, 2, 9, 14, 16, 20, 24, 25 and 27, or an ortholog thereof;
d) a polypeptide comprising an amino acid sequence being identical to or having at least 100% identity with SEQ ID NOs: 1, 2, 9, 14, 16, 20, 24, 25 and 27, or an ortholog thereof;
e) a polypeptide comprising an amino acid sequence which is a variant of the amino acid sequence of (a), (b), (c) or (d); and
f) a polypeptide comprising an amino acid sequence which is a fragment of the amino acid sequence of (a), (b), (c), (d), or (e).

36. An isolated nucleic acid molecule comprising a polynucleotide selected from the group consisting of:
a) a polynucleotide sequence comprising the polynucleotide sequence SEQ ID NOs: 11, 15, 19, 21, 23 and 26;
b) a polynucleotide sequence comprising a fragment or variant of the polynucleotide sequence of SEQ ID NOs: 11, 15, 19, 21, 23 and 26, wherein the polynucleotide sequence initiates transcription in a plant cell;
c) a polynucleotide which is complementary to the polynucleotide of (a) or (b); and;
d) a polynucleotide that hybridizes under stringent conditions to the polynucleotide of (a), (b) or (c).

37. The nucleic acid molecule of claim 36, wherein said polynucleotide initiates transcription in a cell of an ovule primordia, ovule, female sporocyte, female gametophyte, or female gamete.

38. An expression cassette comprising a nucleotide sequence of claim 36 operably linked to a heterologous nucleotide sequence of interest.

39. A plant comprising the expression cassette of claim 38.

40. The plant of claim 39, wherein the plant is maize, wheat, rice, sorghum, barley, oat, lawn grass, rye, soybean, Brassica, sunflower, millet, sugarcane, cotton, safflower, tobacco, or alfalfa.

41. A transgenic seed of the plant of claim 40.

42. The plant of claim 39, wherein the heterologous nucleotide sequence of interest comprises a gene product that confers non-reduction, non-recombination, parthenogenesis, or any combination thereof.

43. A method for expressing a polynucleotide sequence in a plant or a plant cell, said method comprising introducing into the plant or the plant cell an expression cassette comprising a promoter operably linked to a polynucleotide sequence of interest, wherein said promoter comprises any of the polynucleotide sequences of claim 36, wherein said polynucleotide sequence initiates transcription in said plant.

44. The method of claim 43, wherein the promoter expresses the polynucleotide sequence of interest in a ovule primordial, ovule, female sporocyte, female gametophyte or female gamete in the plant or plant cell.

45. The method of claim 43, wherein the polynucleotide sequence of interest comprises a gene product that confers non-reduction, non-recombination, parthenogenesis, or any combination thereof.

46. An isolated polynucleotide comprising a member selected from the group consisting of:
a) a polynucleotide that encodes the polypeptide of SEQ ID NOs: 1, 2, 9, 14, 16, 20, 24, 25 or 27;
b) a polynucleotide comprising the sequence set forth in SEQ ID NOs: 3, 4, 5, 6, 7, 8, 10, 12, 13, 17, 18, 22, 28 or 29;
c) a polynucleotide comprising at least 300 nucleotides in length which hybridizes under stringent conditions to a polynucleotide of (a) or (b), wherein the conditions include hybridization in 40 to 45% formamide, 1 M NaCl, 1% SDS at 37° C. and a wash in 0.5× to 1×SSC at 55 to 60° C.;
d) a polynucleotide having at least 80% sequence identity to SEQ ID NOs: 3, 4, 5, 6, 7, 8, 10, 12, 13, 17, 18, 22, 28 or 29, wherein the % sequence identity is based on the entire coding region and is determined by BLAST 2.0 under default parameters. wherein the polynucleotide encodes a polypeptide that confers Nrf4 activity; and
e) a polynucleotide fully complimentary to a polynucleotide of any one of (a) to (d).

47. An isolated polypeptide selected from the group consisting of:
a) an isolated polypeptide comprising any one of SEQ ID NOs: 1, 2, 9, 14, 16, 20, 24, 25 or 27, wherein said polypeptide confers Nrf4 activity;
b) a polypeptide that is at least 80% identical to the amino acid sequence of any of SEQ ID NOs: 1, 2, 9, 14, 16, 20, 24, 25 or 27, wherein said polypeptide confers Nrf4 activity;
c) a polypeptide that is encoded by a nucleic acid molecule comprising a nucleotide sequence that is at least 80% identical to any one of SEQ ID NOs: 3, 4, 5, 6, 7, 8, 10, 12, 13, 17, 18, 22, 28 or 29 or a complement thereof, wherein said polypeptide confers Nrf4 activity;
d) a polypeptide that is encoded by a nucleic acid molecule that hybridizes with a nucleic acid probe consisting of the nucleotide sequence of any of SEQ ID NOs: 3, 4, 5, 6, 7, 8, 10, 12, 13, 17, 18, 22, 28 or 29, or a complement thereof following at least one wash in 0.2X SSC at 55° C. for 20 minutes, wherein said polypeptide confers Nrf4 activity; and
e) a fragment comprising at least 100 consecutive amino acids of any of SEQ ID NOs: 1, 2, 9, 14, 16, 20, 24, 25 or 27, wherein said polypeptide confers Nrf4 activity.

48. An expression cassette comprising a promoter operably linked to a polynucleotide of claim 46 or a polypeptide encoding a polypeptide of claim 47.

49. The expression cassette of claim 48, wherein the promoter is a heterologous promoter.

50. A plant cell transformed with the expression cassette of claim 48.

51. A plant regenerated from the plant cell of claim 50.

52. The plant of claim 51, wherein the plant is maize, wheat, rice, sorghum, barley, oat, lawn grass, rye, soybean, Brassica, sunflower, millet, sugarcane, cotton, safflower, tobacco, or alfalfa.

53. The seed from the plant of any of the claims 51-52.

54. A method of increasing Nrf4 activity in a plant, the method comprising increasing the expression of one or more Nrf4 genes in a plant cell by introducing into the plant any of the polynucleotides in claim 46, any of the polypeptides of claim 47, or the expression cassette of claim 48.


SEQUENCE LISTING (37 sequences)

SEQ ID NO: 1 - protein, 248 aa, Zea mays
MGTEYPTFADVSGARALLFLADSTPRLAPPSRPPALSEEFYCYSGSSSSYSGASTRSCVSDSAQRGRPVDPLRVLSVVASLRRIDPKVLAEATSALFHTDAEKKRKGVWIEIDSGGDDGQSERSSAVASEGSTVTAAASAGSTATSGRCRGAPRVGCAAGGKGPRRAEVIMQWFSQTQAGPATENDIRAAVGDNSGTSKAIRWLLKQEGGLRRAGNGGALDPYVYMAFTSLGGSPWLAVLQPGPHCCR

SEQ ID NO: 2 - protein, 235 aa, Zea mays
MGTEYPTFADVSGARALLFLADSTPRLAPPSRPPALSEEFYCYSGSSSSYSGASTRSCVSDSAQRGRPVDPLRVLSVVASLRRIDPKVLAEATSALFHTDAEKKRKGVWIEIDSGGDDGQSERSSAVASEGSTVTAAASAGSTATSGRCRGAPRVGCAAGGKGPRRAEVIMQWFSQTQAGPATENDIRAAVGDNSGTSKAIRWLLKQEGGLRRAGNGGALDPYVYMVADRSDEVN

SEQ ID NO: 3 - DNA, 1342 bp, Zea mays
  source 1..1342: mol_type = other DNA
cttttcgaaaatcccccacagaatcccatccccccgggccccggcaaccgctggcgctgcctccagccctccaacaccacgaccgccgggcggctcctcctcctctgccggagagctcggcgggcggcatgggaacagagtatcccaccttcgccgacgtatccggggcccgcgcgctcctcttcctcgccgactccacaccccggctggccccgccttcgcgtcctcccgctctcagcgaggagttctactgctactcgggctcctcgtcctcctactccggcgcgtcgacgaggtcatgcgtctccgactcggcacagcgcggtcgccccgtcgatcccctacgcgtgctctccgtcgtcgcctccctccgccgcatcgaccctaaggtgctagccgaggccacaagcgcgctgttccataccgacgcagagaagaagcggaagggcgtgtggatcgagatcgacagcggtggtgacgacggccagagcgagaggagcagcgccgtggccagcgaggggagcaccgtcacggccgccgcgtccgcgggctccacggccacgtcggggagatgccgcggggctccgcgggtgggttgtgctgctggtggtaaggggccgaggagggcggaggtgatcatgcagtggttttcgcagacgcaagctgggccagcgacggagaacgacatccgcgccgccgtcggcgacaactccggcacgagcaaagcgatacgatggctgttgaagcaggagggcggcttgcggcgtgcaggcaatggtggtgccctggatccgtatgtttatatggcgtttaccagcctaggtggaagtccctggctcgctgtgctgcagcctggtccgcattgctgtcgctgaaagctgcatgagaaaggatgagatggtgtgcgacaaggtacggctccgtggtggagttgcctccggcggggcacccggaacaccttcccggtgcgtgcaagccattgtcgtagcccagagcagcgacgcacctcggctcgttgaaggtaaaccagttcttcaccctgtctccgaacgcgtggaagcagaactcggcgtagtctgcaaacgcctcccttcgattgggcacggcgtgcagctgggtcagactgactgacacatggttgaatcaatttgaaatttggtttctagatgtcgtctagtttgcaattcttacacaatctttgggctaagccagcccaggtactgttcatggagtgccaatgggaggtcataatggtagagatttgcatacggcgcgatacctgacttgtatgtatacatatatataaaggttaaccaggaatctgaatgtttcctgaaagaat

SEQ ID NO: 4 - DNA, 747 bp, Zea mays
  source 1..747: mol_type = other DNA
atgggaacagagtatcccaccttcgccgacgtatccggggcccgcgcgctcctcttcctcgccgactccacaccccggctggccccgccttcgcgtcctcccgctctcagcgaggagttctactgctactcgggctcctcgtcctcctactccggcgcgtcgacgaggtcatgcgtctccgactcggcacagcgcggtcgccccgtcgatcccctacgcgtgctctccgtcgtcgcctccctccgccgcatcgaccctaaggtgctagccgaggccacaagcgcgctgttccataccgacgcagagaagaagcggaagggcgtgtggatcgagatcgacagcggtggtgacgacggccagagcgagaggagcagcgccgtggccagcgaggggagcaccgtcacggccgccgcgtccgcgggctccacggccacgtcggggagatgccgcggggctccgcgggtgggttgtgctgctggtggtaaggggccgaggagggcggaggtgatcatgcagtggttttcgcagacgcaagctgggccagcgacggagaacgacatccgcgccgccgtcggcgacaactccggcacgagcaaagcgatacgatggctgttgaagcaggagggcggcttgcggcgtgcaggcaatggtggtgccctggatccgtatgtttatatggcgtttaccagcctaggtggaagtccctggctcgctgtgctgcagcctggtccgcattgctgtcgctga

SEQ ID NO: 5 - DNA, 708 bp, Zea mays
  source 1..708: mol_type = other DNA
atgggaacagagtatcccaccttcgccgacgtatccggggcccgcgcgctcctcttcctcgccgactccacaccccggctggccccgccttcgcgtcctcccgctctcagcgaggagttctactgctactcgggctcctcgtcctcctactccggcgcgtcgacgaggtcatgcgtctccgactcggcacagcgcggtcgccccgtcgatcccctacgcgtgctctccgtcgtcgcctccctccgccgcatcgaccctaaggtgctagccgaggccacaagcgcgctgttccataccgacgcagagaagaagcggaagggcgtgtggatcgagatcgacagcggtggtgacgacggccagagcgagaggagcagcgccgtggccagcgaggggagcaccgtcacggccgccgcgtccgcgggctccacggccacgtcggggagatgccgcggggctccgcgggtgggttgtgctgctggtggtaaggggccgaggagggcggaggtgatcatgcagtggttttcgcagacgcaagctgggccagcgacggagaacgacatccgcgccgccgtcggcgacaactccggcacgagcaaagcgatacgatggctgttgaagcaggagggcggcttgcggcgtgcaggcaatggtggtgccctggatccgtatgtttatatggtcgcagacagatcagatgaagttaactaa

SEQ ID NO: 6 - DNA, 1625 bp, Zea mays
  source 1..1625: mol_type = other DNA
aggaccgataaaacgtcatgtacgctaaatacagcgcacggagacattcgctatgcgcccaataataaccgctaaaccagctggtaaaatataccgggccttcttcgcctctacatctagcggacgagcgaccgctcgctatgcactgttcgaacgtgtgctccgcctcctcctgttccctcccgcgcttgctcggaagctatagcgaccgaccctgactcagacgatcggcgtcgtcgtgatcgtcgcggctacgacggcagagaagcgcgtctggcccgatgtcgcgactgggggtatagatggccatttggcactaacacgatgggtcagcccaggcacggcacgaaaaagcacggtccaggcacgacccggtccggttagtatagtgccagtgtctggcacggcacggctatagtgccgtgcctgggccactatctcgacccgtagtgctggcacaggcacgacacggttacgtttttttattttaaaaataatagtttacatatattaagtataagaattcaacatataacacaataaaactgcagctgcattggttagatagatacgtttaagcctctcatgcactaggttgtgggtttgagcccccacaactacacattttttgctaaattctataatatactcagatgggccatagtgccaccgggccggcccggcacgactagcaggctcacgggccgtgcctgggccgtggcagcggcacgccggcccatctaggcacggcagttttgtagccgggcctggcgggcacgccagcccgtttggccatatataactgggggcgacgacggcaaggcgaaagttggaagagccatggcggagaaaccgaagctagcgacgatgacggcagacgaggagcgcgcttggcccagtctcacgggctcttcctagccgtcgtacagtgctttcgggagcttgtgtcggccacctacaacttcgatttcttggacacgtttggaaagaggtggcgttgtggtggttgcacctctcgtacattgcaatgcacatgttcgacaatatgtctgagacatgaacgatgtctttatgtgtccaaaacgaatttcttttacaaaacgtaagtcagatagttctttagcaaaaacgattagtactgtatattacataatataattattttatttattgcatagttccattttggtaacataggaacgtttttttacaagtgactgatttaaatacaatatatattgtggaatttggtaacactctagttatacaggatcgaatttagagaatgttgctgaagataaaaaagatataaatgacagaatcttttatagaaaactataaaaaaataaaaaatatttatttaaaaataaaatttaaacgatattgctggagaacaaaccaacagtccaacgatagttagacttagactgaaactgcgggcctctttcccggacaattttcaatgtcatgggccggattcgaatctgaattgggcccatcaatttaagaggcgaaacacgggcccgcaccgccgcacgccttcggagtttccgcctttcccccagtcgctcgaaattttaaaatggcgctgctcccctgtcatcctcacggccacgcccccgaaa

SEQ ID NO: 7 - DNA, 4377 bp, Zea mays
  source 1..4377: mol_type = other DNA
cttttcgaaaatcccccacagaatcccatccccccgggccccggcaaccgctggcgctgcctccagccctccaacaccacgaccgccgggcggctcctcctcctctgccggagagctcggcgggcggcatgggaacagagtatcccaccttcgccgacgtatccggggcccgcgcgctcctcttcctcgccgactccacaccccggctggccccgccttcgcgtcctcccgctctcaggtacctgcctcgcgtcatctgctcatcgcgctacggaattgcacttcatcaccgcaatttcacccgaccctccgttgcggcgcgcttctctgcattgcagcgaggagttctactgctactcgggctcctcgtcctcctactccggcgcgtcgacgaggtcatgcgtctccgactcggcacagcgcggtcgccccgtcgatcccctacgcgtgctctccgtcgtcgcctccctccgccgcatcgaccctaaggtgtgcaatcggcgagttcatcgactcgtgatcgtgatcagcacgcggttgcgagaatatctcttctgcgattacgcactgatggctctgctccttggctccaggtgctagccgaggccacaagcgcgctgttccataccgacgcagagaagaagcggaagggcgtgtggatcgagatcgacagcggtggtgacgacggccagagcgagaggagcagcgccgtggccagcgaggggagcaccgtcacggccgccgcgtccgcgggctccacggccacgtcggggagatgccgcggggctccgcgggtgggttgtgctgctggtggtaaggggccgaggagggcggaggtgatcatgcagtggttttcgcagacgcaagctgggccagcgacggagaacgacatccgcgccgccgtcggcgacaactccggcacgagcaaagcgatacgatggtgagcttgactctgcctccaacttccatttgaaaaggttttgcaatggttacctgattacttctgcctgacctgtgtatccgcgtatctgtgtgcttctgctcatgcaaaacttgcaggctgttgaagcaggagggcggcttgcggcgtgcaggcaatggtggtgccctggatccgtatgtttatatggtatgttaaacttgagcggactacttgaattaggatggtagttggaattgcatttttaattccggaacatgtactgcgcagctaagaacttcaaatcagcgcaaaatgtaagaattcctagaactgcaaattaggatttaggaacttgccacaagcacgactaagcaatttaaccatatcttagttttcctgaaatgcattatatagttaaatttctgaataaattttcaggtcgcagacagatcagatgaagttaactaagctttgtttatagaaatagcgatggagatgtttactgtaaggcaattggacaggccaagttatcttctgaagtgtatctataataatttctacatgctgttgtcttaatgacctggtttccccctaccacagataagtataggatagaagttaagttgtcatgttagcacggtgaagataagctcttgaacagaaaaacggcgagatagaatctcaagtataagttcagtatataaagtatagcgatgaagctaccatgatttcgttgttgatttgagctatataccgtgtggtcaaatatatatggtttggctaaatggattatcgaataaaatgagttcaattttcagcttgttgaatgtaacgaagttttaattcgccccttttgttttttttatgccgtttatgttggtgaacttgtgtgtcatttgtctgtgatcccgtcattgtgcttctacttgttctattatggagactcaaggttttcgaagataccaatttattgccttgcaacaattaacgaagctagagatgtcgagccttgagactcaattgtctgcacggttccttaagctaaatctgacatactaagaggatttgcagagtgaacttttgtatgacacgtgaagccagtggtcctcttgtctgcaattctctagctccttttcttggacagcatatgcttgaaccagaaagctgaatccttggggtacctcttcagagtcttgtagtccacgtacaccaagccaaaccgcgaagtgtacccaagcctccactcgaagttgtcaagcagcgaccacgcaaagtacccaatgactctggcaccatcatctattgccttcttgagctcagttatgtagtctctgtaataacggattcttactgtgtcatgcacaccctgagtaatactgacatcaccaggttggtccattcctgcaagagcacaccacttaattaattcaaagcatataccgttctacctggaaatttgaatttcgattcatgagttaccgttttcagcaaggatcattgtaggatttttgtaagtttccttgacatagctgacagccttgttgatgccccacggcacaatgtacagccagtaggagtttgcctacagagctcaaagaaatttacagtaccataatcagaggcaaaccaactttcctgctttcgacgtctgaagacatctagcagtacacttacgtgagcgccaataggaactccatttcgttcgtctgcagaaacatgtgaggatcaacatacgactagatgaggtgacggcagcacaagaaactgcggaattcaagaatttggttcgtggagtcagaaacttacagacaaaaccaacatgccaatcatcctggtagctgactggtgtcaggttccatgtcccagggtccttcatgtagaaagaagtgtagtggttgatgccaacatagtctatagagcctttcaccatcctggcttcttcatcgctgaacaacggtagcctgtctttggcaatctcttgcatcgagtacgggtaccgtccatgtacaatggggtcaaggaacctgtaaggatgttgcagtatcagtttgttcttggcaacgagctgtgttggtttgtttccccaaatataagatatcatcagtttgtgtctagttgcctagtacactatagctaataatgtcactgatacaaagaacctctaataagatcagcaatgacaattgggtccttgtataatcaagtgcatgtgctttcaggcgtttaccagcctaggtggaagtccctggctcgctgtgctgcagcctggtccgcattgctgtcgctgaaaggttcgtaccacacgaaatccaggagaattccaatcttccccttctggtgaagctgttcgtttagtggaaaaggaatacggatcattttctggatgcctgaacactgacattttaattactgaactaaatctgcaccgtgcaattcacacttctggttctgggttcggaggcttgaaatatatagttagcaggtctgcattttatttttacctgatacttgtcgcggtatcgcctgacagcagctgcatgagaaaggatgagatggtgtgcgacaaggtacggctccgtggtggagttgcctccggcggggcacccggaacaccttcccggtgcgtgcaagccattgtcgtagcccagagcagcgacgcacctcggctcgttgaaggtaaaccagttcttcaccctgtctccgaacgcgtggaagcagaactcggcgtagtctgcaaacgcctcccttcgattgggcacggcgtgcagctgggtcagactgactgacacatggttgaatcaatttgaaatttggtttctagatgtcgtctagtttgcaattcttacacaatctttgggctaagccagcccaggtactgttcatggagtgccaatgggaggtcataatggtagagatttgcatacggcgcgatacctgacttgtatgtatacatatatataaaggttaaccaggaatctgaatgtttcctgaaagaatatatctatatctatatacataaataactgcctaactgccatatggtggaaacttaccttgctggagcatgtaatctatgagcctgttgtagtaatccactccttcctggtttaccttgccagttccatctgcgtatattatatatacacacagaactctacctcagaaactccttggacccaagaaaattctacagaatggagcacacctacctgggaaaatcctcgaccaagagatcgaaaatcggtacgcatcaaagcccatgttcttcattatgttcacatcttcctgttcctcgaagaacatccgtcaggcgtcacgaaacaacaaggagcacccaaaaacaagaagaacccaatgcattcagagaacacaccttgtaccgatgatactcgtcgaccgtcacgtcagcggtggcattgttagggatggtcccttgtttttttcattccccggat

SEQ ID NO: 8 - DNA, 654 bp, Brachypodium distachyon
  source 1..654: mol_type = other DNA
atgggttccgctcccgccgccgcggccttcccgacctactccgacttcgccggcgccggcgcgctgctctccctcgccgactcctcgccctcgccacccctccccaccctcaggtcgtgcgtgtccgactcgatccggcgcggccgccccgtcgaccccctccgcgtgctcgccgtcgtcgcctccctccgccgcatcgaccccaaggtgctggccaaggcgaccagtaagctgtgccggggcgaggaggcgaagaagcggaagggcttgtggatcgagatcagcgacgaggaggaggaggacgacgacgacagcgagaggggcagcgcggtggccagcgaggggagcacgatcaccggcgcggcgtccgccggatccacggccacgtcgggaggatgccgccggcctccgcgggcgagcgggggcggcgagaatctgccgcggagggcggactcgatcatggagtggctatcgcggccgaaggcggcgccggccacggagacggccatccgcgccgccgtcggcgacaacgccgtcaccagcaaggcgctgcgctggcttctgaagcagccgagaggcctgcgccgtacaggcagcggtggtcgtgcagatccatttgagtacatggtcctcgagtactttgatcacgcctga

SEQ ID NO: 9 - protein, 217 aa, Brachypodium distachyon
MGSAPAAAAFPTYSDFAGAGALLSLADSSPSPPLPTLRSCVSDSIRRGRPVDPLRVLAVVASLRRIDPKVLAKATSKLCRGEEAKKRKGLWIEISDEEEEDDDDSERGSAVASEGSTITGAASAGSTATSGGCRRPPRASGGGENLPRRADSIMEWLSRPKAAPATETAIRAAVGDNAVTSKALRWLLKQPRGLRRTGSGGRADPFEYMVLEYFDHA

SEQ ID NO: 10 - DNA, 1359 bp, Brachypodium distachyon
  source 1..1359: mol_type = other DNA
atgggttccgctcccgccgccgcggccttcccgacctactccgacttcgccggcgccggcgcgctgctctccctcgccgactcctcgccctcgccacccctccccaccctcaggtacgtgcggctcctgggttggattattctgcgcgcgcgcacttccgagcgcgagacgtactcaccgctcccccgcgcatacatttgcagcgacgagctctcgtcctactcgggctcctcctcctcctactccgtcacctccggcaggtcgtgcgtgtccgactcgatccggcgcggccgccccgtcgaccccctccgcgtgctcgccgtcgtcgcctccctccgccgcatcgaccccaaggtactgaacaagcagccgctcatctgctaattagcgattcaatttggccgtagtgaataatgggggctcggctcgtttgcgctctaggtgctggccaaggcgaccagtaagctgtgccggggcgaggaggcgaagaagcggaagggcttgtggatcgagatcagcgacgaggaggaggaggacgacgacgacagcgagaggggcagcgcggtggccagcgaggggagcacgatcaccggcgcggcgtccgccggatccacggccacgtcgggaggatgccgccggcctccgcgggcgagcgggggcggcgagaatctgccgcggagggcggactcgatcatggagtggctatcgcggccgaaggcggcgccggccacggagacggccatccgcgccgccgtcggcgacaacgccgtcaccagcaaggcgctgcgctggtgagcacaacgcttcctccaatccaattatcgtctgaagaggttttggaatggttgccctgattgctccttctgtgctttgttcgtgcgtctctctgttcgaaatttgcaggcttctgaagcagccgagaggcctgcgccgtacaggcagcggtggtcgtgcagatccatttgagtacatggtacgtgaaatttgtgccgacagagttcgcattggaatgtgaaatgtcaaattcgaaatatattatgactgtttgaatgataatttctcaaggtagcttaacagaacttgagactggatcagtctgaccagcgtgcaaaattcagttatgagtcagtgaggtgttacttgttagcaattttcagttacccagtagagagcagtggaattttaaagcaatcgttaaacctgataaagtttgtgttagttcaagtgatcacctggcttgtataattatggaaggtctattagttgaatcatgaccactgcaaatttggagcaattatgatgtctaattagtaaatcaatttggtatcccaggtcctcgagtactttgatcacgcctga

SEQ ID NO: 11 - DNA, 1700 bp, Brachypodium distachyon
  source 1..1700: mol_type = other DNA
ttagtacttgccagtggtgtgggagtcaacgccggcgcggcgagcgacgccggtggcgcagcccggggtcaggtcagttggtccggccggcgattgggtttgttcggggaggggattttaccattttggacccgagctaagctgctatggtgggcctttgctgagctgagctgcttgctctctcttgtgaaattccgaaggcgtcgacgaaattttttagttccgaatccaccgtcatggcctctcaaatctcaaaatttagaagttcgatttgtttaccggtttttctcttactaagaagaaaatttacgagttgattagaagcttcaagaccatcctaggcctgtaataaagagagcttttcgatgcacacactttgtatttggtgttcatgactcgtgtgttcttgtttgcttacgtaaaagggaaaatgaatatgaggtcaaacttatttgaacgggcacttccgttttgtagtacactcatttcttatgatgtaagatttgaattatgtttaactgtcgtagcgaggaccatgactcgcaattgtaatcttctgaggatgcaaactatggaaatgtggagtattacattgcataatcatcttcagaatgattggcgtctgaaagtacaccggcattaaccatgagcgaaaatagaaaataacaggagactatataaagtgcatcaacgaaggtggaccgatattgactattgactctccatgtcacctatatcggcaataaaatgacttatataatgtgttgtgtctttagattggtcatgaactttgttggcataaaattcagtgataacaaggcttactcttggctagggagtcaattctcttctctcttctccacgccaacaattttcatatcaaaatcaacatttgttgatactctaatttgcaattgagttcgcattgcgcttttttcaagatgttggatcaaatctttttattttcctctaacatactcccactcgccgccgtctcaaacaatgtggtgccgggaagtttcagtcaaaaaattccatctctaagccttcattagccccatgctcctggtggtattttcaaaggtagatttttaattggaaatggtcaaatggaggagagggagaagagaaatcgacttttgtcgaaaaatcaaagtctccagaattcggccaaaagtgtgagctaagaatcaaccttgttatgccctctcctccatttggccaaaagtgtgagctaagaatcacttacaccatcgaatagagcaagtgatggtaatattgactttactgttgtgtatgcttttaaaacccaaattatgccttctccggcttaccataaaaggcaggtcgaagaagcaaggaaagaagacgatttcattaaacaaatcgagtgttcatcaataaatcggaaacgaataaactgattgatcgcatactgaaacaggatcaaaacacgcaatacacacctcgctttacaaactcatcctggcggccccacatgtcagcctgccctatggacctccgccctcacgctcgaaaattcaaaaaacgggaaaggcaacacaacacggctcgtttaaaatccgagagatcagccgcagcatcccaaactcccttcccaaatccccaaacagaacaaaaatcccaactgcaagagcagcagcctctgctgcctcctctctcccgaccgcaccaccacg

SEQ ID NO: 12 - DNA, 957 bp, Sorghum bicolor
  source 1..957: mol_type = other DNA
atgggccggattcgaatcttgattcggcccatcaatttaagaggcgaacccggacccgcaccggcgttccggcttcccccagacgctcgaaatttcaaaatggcgctgctcccctgtcagcctcacggccacgccaccgaaacttttcgaaaatcccccacaaaatcccagcggccagctccccgaggccccggcaaccgctgcctccgccaccaccgggcgactgctccacctctgccggagagcgcggcgggcggcatgggagcggagtatcccaccttcgaagaggtctccgcggcccgcgtcctcctcctcctcgccgactccacgcccccaccggccctgccttcgcctcctcccacgctcagcgacgagttcttctgctactcgggctcctcatcctcctactccggcgtgtcggcgaggtcgtgcgtctccgactcggcgcagcgcggccgccccgtcgaccccctccgcgtgctctctgtcgtcgcctccctccgccgcctcgaccctaaggtgctcgttgaggccacaagcgcgctgttccataccgacaaagagaagaagcggaagggcgtgtggatcgagatcgacagcggcgatgacgaggatgaccagagcgagaggagcagcgccgtggccagcgaggggagcaccgtcacggccgccgcgtccgcgggctccacggccacgtcggggagatgccgccaggctccgcgggtgggttgtgctgctggtgggactgggaaggggctgaggagggcggacgtgatcatgcagtggttttcacggccgcaagctgggccagccaccgagaatgacatccgcgccgccgtcggcgacaactccggcacgagcaaggcaatacgctggctgctgaagcaggagggcggcttgcggcgtgcaggcactggtggttccctggatccgtatgtttacatggtcgcagactga

SEQ ID NO: 13 - DNA, 1952 bp, Sorghum bicolor
  source 1..1952: mol_type = other DNA
gccgtggactgcgtggtcttgggggtgactgggtgagtaggctctaacggatgttggacttggaccgaactgcaggcctcttttctgggcaattctcactgccatgggccggattcgaatcttgattcggcccatcaatttaagaggcgaacccggacccgcaccggcgttccggcttcccccagacgctcgaaatttcaaaatggcgctgctcccctgtcagcctcacggccacgccaccgaaacttttcgaaaatcccccacaaaatcccagcggccagctccccgaggccccggcaaccgctgcctccgccaccaccgggcgactgctccacctctgccggagagcgcggcgggcggcatgggagcggagtatcccaccttcgaagaggtctccgcggcccgcgtcctcctcctcctcgccgactccacgcccccaccggccctgccttcgcctcctcccacgctcaggtactttcctcgcctgtctcgcgtcatctgctcatcgcaccaccgaattgcacttcatcatcgcaagttaaccgagcctccgttgcggcgcgcttctccgcattacagcgacgagttcttctgctactcgggctcctcatcctcctactccggcgtgtcggcgaggtcgtgcgtctccgactcggcgcagcgcggccgccccgtcgaccccctccgcgtgctctctgtcgtcgcctccctccgccgcctcgaccctaaggtgtgcaatccggttcctcgattgatgatcagcacgcggttacgagcagcctcctctgcagttacgcactgatggctctgttccttggctccaggtgctcgttgaggccacaagcgcgctgttccataccgacaaagagaagaagcggaagggcgtgtggatcgagatcgacagcggcgatgacgaggatgaccagagcgagaggagcagcgccgtggccagcgaggggagcaccgtcacggccgccgcgtccgcgggctccacggccacgtcggggagatgccgccaggctccgcgggtgggttgtgctgctggtgggactgggaaggggctgaggagggcggacgtgatcatgcagtggttttcacggccgcaagctgggccagccaccgagaatgacatccgcgccgccgtcggcgacaactccggcacgagcaaggcaatacgctggtgagcttgactctgcctctgacttccattttaagcggttttgcagtggttgcctgattgctgcctaacctgtgtatctgcgtgcttcctttcgtgcaaaatttgcaggctgctgaagcaggagggcggcttgcggcgtgcaggcactggtggttccctggatccgtatgtttacatggtatgttaaactcgtgcagactacttgaattaggatggtagttggatttgcattttaaaatccacaatgtgtagtgcacacacagctaagaacttcaaatcagtgaattgaaatgtagaattcccataactgcaaagtaggatttaggaacttgccggacgcacgagaagtatatcttaccttttctgatgtgcattacagttattgactcgcatgatatgtcatgtatactaactttctgaataaattttcaggtcgcagactgatccgatgaagttaactaagttttgtttatagcaatggagatgtttactgtaaggctattgcagacgccaagttatcttctgaaatgtatctagtaatttctacctgctgttgtcttaatggccgggttttgccttcacacagaagtataggatagaagttaagttgtcatgttagcacgatgaagataagctcttggacagaaaaatggcaagatagaatctcatgtataaatttagtatagaaagtacagcaatgaagatagcatgctttcgctgttgatttatgctgagacgatatatcgtataaatttagtatcgaataaaatga

SEQ ID NO: 14 - protein, 318 aa, Sorghum bicolor
MGRIRILIRPINLRGEPGPAPAFRLPPDARNFKMALLPCQPHGHATETFRKSPTKSQRPAPRGPGNRCLRHHRATAPPLPESAAGGMGAEYPTFEEVSAARVLLLLADSTPPPALPSPPPTLSDEFFCYSGSSSSYSGVSARSCVSDSAQRGRPVDPLRVLSVVASLRRLDPKVLVEATSALFHTDKEKKRKGVWIEIDSGDDEDDQSERSSAVASEGSTVTAAASAGSTATSGRCRQAPRVGCAAGGTGKGLRRADVIMQWFSRPQAGPATENDIRAAVGDNSGTSKAIRWLLKQEGGLRRAGTGGSLDPYVYMVAD

SEQ ID NO: 15 - DNA, 1700 bp, Sorghum bicolor
  source 1..1700: mol_type = other DNA
aatctcttcaaccccaagatgcactgaaggcagcttctttggtgcaaactccttcttgtccccatgtccaatggttgccctaggccggtcaatctccaatgacatcaaccgtggctgccggtacccgatagcaacaagatcttggagccaagaagtcatgaaatgcaaatccttttcaccccagaatccctcctctgtcatcgcatagcttagatcaagaatccgctcgaatagtgtcctcttactggatcgccactccattaggaactttatcagcttccctacattgacatggagatccttctcttcatcaaatgtgtgagcacgcacattatctatgcggtgcacagttggtgggtagacaactaggtaccctccaatctcccatagtatcctctgcgcccagtatccgcgtatgacatccgatgccattggactcactgacactggcaatgcaagcccccagaacgccggcgagtggaacaatgtgttaacagagttgactggcgtcatcgtgccctgtggtagcgcaaccttgggtgcatccgcgtcaaaccggaaatcaaatgcctccatctccaatgacttccgggtgaagtagaacactgagtcaacatctggaaggccattacacattccttgctgcatgaattggccaccactgaatatctcagtgtagaactcctcagcatccagttcccctgccttctccagcggcagcccccgtggccagaccgacggctgcccaaagtgcacgaacgggttcacgaccgtccggttaggatcggcatggctgtactgcagcagcacggcgcctccctggcgctggtccagatcgacatcgaagtgactggtgaggttgccgcccaggacggcgttgcgcacgtcggcgtcgtagatgacccgcgccccgcgctgcacggcgaagaggtacgccgcggccttgcgcgccgggccccgggctggaaggaaagcgacggagcggaatccgaggtgatcctggtcggcgagcgtgagcaggacggcgcctgggtgggaccaatccgcgggggtcgcctcgtcggcgaccgcgaggagctgccacccggggaccgcgcgcagcgggcggtgccgcggggcatgcggcgacgcgatgaagacgatccagcgagagccgcggaggtccgggtgcggcgaggagaggaggacggggagcgggggcaccttggaccagagcacggacgggtacgggaggcggcggggcgcgcggtaggcggcggggcagagcgcggagggggaggcgccgccgtagaggaggagcagcaggaacggcgccgtggcaagcgccgcgaggaggacgtagacgacgcgcctagatgcgggtctggaggtagtgggcaaggcgttcggtggcatttgggcgttggaggagaaaaggggctggctcgacgacgccgggtccgccggtgagattcggcacttcggccggcgaggggaccggaagggcggacggcgtggagccgtggactgcgtggtcttgggggtgactgggtgagtaggctctaacggatgttggacttggaccgaactgcaggcctcttttctgggcaattctcactgccatgggccggattcgaatcttgattcggcccatcaatttaagaggcgaacccggacccgcaccggcgttccggcttcccccagacgctcgaaatttcaaa

SEQ ID NO: 16 - protein, 263 aa, Panicum virgatum
LRPTPPKLFENPSTGYTPASNSRRPLLLFCRLVGGMGADYPNFADVSGASALLFLADSSPAPPPPPPALSDEISCHSGSSSYSGASASSCVSDSARRGRPVDPLRVLAVVASLRRINPKVLAEATSTLFHSGAEKKRKGVWIDIDSYYDDADQSERSSAVASEGSTVTAATSVGSTATSGRCRRPPRASDCGGGEKPPRRADVIMQWFSRSQAGPATENDIRAAVGDNSGTSKAIRWLLKQKGGLRRAGTGGPLDPYVYMVAG

SEQ ID NO: 17 - DNA, 792 bp, Panicum virgatum
  source 1..792: mol_type = other DNA
ctcaggcccacaccaccgaaacttttcgaaaatcccagcacgggctacacccctgcctccaactcccggcgacctctcctcctcttttgccggctcgtcggcggcatgggagcggactaccccaacttcgcggacgtctccggggccagcgccctcctcttcctcgccgactcctcgcccgcaccgcctccgcctcctcccgctctcagcgacgagatctcctgtcactcgggctcctcctcctactccggagcgtcggcgagctcgtgcgtttccgactcggcaaggcgcggccgccccgtcgatcccctccgcgtgctagccgttgtcgcctccctccgccgcatcaaccctaaggtgctcgccgaggccacaagcacgctgttccacagcggggcggagaagaagcggaagggtgtgtggattgatatcgacagctactacgacgacgcggaccagagcgagaggagcagcgcggtggctagtgaggggagcaccgtcacggccgccacgtccgtgggctccacggccacgtcgggtagatgccgccggcctccgcgggcgagtgattgtggtggtggtgagaagccgccgaggagggcagacgtgatcatgcagtggttttcgcggtcgcaagctgggccggccacggagaacgatatccgcgccgcggttggggacaactccgggacaagcaaagcgatacgctggctgctgaagcagaaggggggcttgcggcgagcaggcactggcggccccttggatccatatgtttatatggtcgcaggctga

SEQ ID NO: 18 - DNA, 2009 bp, Panicum virgatum
  source 1..2009: mol_type = other DNA
ctcaggcccacaccaccgaaacttttcgaaaatcccagcacgggctacacccctgcctccaactcccggcgacctctcctcctcttttgccggctcgtcggcggcatgggagcggactaccccaacttcgcggacgtctccggggccagcgccctcctcttcctcgccgactcctcgcccgcaccgcctccgcctcctcccgctctcaggtacgttcctccccttcctctcgtcgtaccgccgaattgcgtgaccgcacgcactccgtcgttgcaatttaatcgaccctccgttgtgctgcgcatctgcacattgcagcgacgagatctcctgtcactcgggctcctcctcctactccggagcgtcggcgagctcgtgcgtttccgactcggcaaggcgcggccgccccgtcgatcccctccgcgtgctagccgttgtcgcctccctccgccgcatcaaccctaaggtgagcaatccgattcctcgattgctgatcagcgcgcgattgcaagccgtctcttccgcagttacgcactgaatactgatggctctgctcgttggctccaggtgctcgccgaggccacaagcacgctgttccacagcggggcggagaagaagcggaagggtgtgtggattgatatcgacagctactacgacgacgcggaccagagcgagaggagcagcgcggtggctagtgaggggagcaccgtcacggccgccacgtccgtgggctccacggccacgtcgggtagatgccgccggcctccgcgggcgagtgattgtggtggtggtgagaagccgccgaggagggcagacgtgatcatgcagtggttttcgcggtcgcaagctgggccggccacggagaacgatatccgcgccgcggttggggacaactccgggacaagcaaagcgatacgctggtgagcaggactttgcctccaacacccattttaagcggttttgaaatggttgcctgattgctgcctgaacatgtgtatctgcttgcttctgttcgtgcgaaatttgcaggctgctgaagcagaaggggggcttgcggcgagcaggcactggcggccccttggatccatatgtttatatggtaagataagattcgtgcaagagtacttaaattaggatggtagttggaattggattttaaattcatatgtgttgtgttgagtgaagattgctagtttgttttagaaattttgaattggatctgtatggacctgggttattgtgcaaattttgagtggagacagtgatcagttagctattcacttcttggaatactatacttcactagtatgaagtggcgcagcataggatgacataactgattcaagtgtacactgatcattcttgactttaatatttctagaaattttactgcaaatttcagagatattgtggtctggtagaaaagttgaaaccctgaatatctttgcattttggttaactgtggagtttgtaaaaatgcttgtaggagactagttgtgagataagagcttttgaattgtgagattcagatactgggtctataaggtcttgtgtcagtgtgcaaagttcggattgaaagagttagcaattagcattttgtagtgacccaatatggaataaacatttgaaaagttaagatgaactgctgaaaatgatttgtgcttatcctgctttacaaagatcttacttgactttatagttctgaaggttctatagattgaattatcagctcttatttcttgaatacttaaattatcagctctgtaccacatcggaaagttgaacacctgaaggccaatgcacatccaggaaggtgaaaccaatgaaatgtataatttataaaagtgatgtggttgtcgcagcatagtgtactgttgcaagctcgaatagcagttccaaccacatcctgagttttttaaattttgcagaatatgtattttatagttattaacccacataatttatcgtgtataataactttttgaatgaaatttcaggtcgcaggctga

SEQ ID NO: 19 - DNA, 1700 bp, Panicum virgatum
  source 1..1700: mol_type = other DNA
cttggggacaaactcctccttgtccccatgtccaattgttgcccttggccggtcaatatccagtgacagcaaccgtggttgccggtacccaatagcaaccagatcttgcagccacgcagccatgaaatgcaaatccttttcaccccagaacccttcctctgtcattgcatagctcaaatcaagaatgcgctcaaacagtgtccgcttactggatcgccactccattaggaactttatcaacctccctatattgacatggatgtccttctcatcatcaaatgggtgagcatgcacattatcgatccggtgtaccgttggtggatagaccactaggtaccctccaatctcccatagtatcctttgcgcccagtatccacgtatgacatctgatgccattggactcactgagactgacaatgcaagcccccagaatgccggtgagtggaacaatgtattaacagagttgatgggtgccatcatgccctgcggcagcgcgaccttaggtgcatacacatcaaactggaaatcgaatgcctccatttctaaagacttccgggtgaagtagaacactgcgtcaatatctggaaggccattgcacatcccctgctgtatgaattgcccaccgctgaatatctcagtgtagaaatcctcagcgtccacctcaccggccttctccagcggcagaccccgcggccagaccgacggctgcccaaagtgcacgaacgggttcacgaccttccggttaggatcggcgtggctgtactgcagcagcacggcgcctccctgccgctggtccagattgacatcgaagtgcttggtgaggttcccacccaggacggcgttgcgcgcgtccgcgtcgtagatgacctgcgccccacgctgcacggcgaagaggtacgccgcggccttgcgcgccggccctcgcgcggggaggaaggcgacggagcggaagccgaggcgggcctggtcggcgagcgtgagcagcacagagcccgggtgggaccagtccgctggggtcgccacgtcggcgacggcgaggagctgccacccggggaccgcccgcagcgggcggtgcttcggggcgtgcggtgacgcggagaagacgatccagcgggaggcgcggagggccgggtgcggcgaggtggggagcgccgggagtgggggaacgcgggaccagaccacggatgggtaggggaggcggcgtggggcgcggaacgaggcggggcagagcgcggtgggggaggcgccgccgtagagaaggaggagaaggaagggcgccgaggcgagcgcggcgaggaggacgtaggcgacgcgcttggaggcgcgcggtggcattttggggagggaaagggtgaggtgacctgatgatgtgccagggcctagggaggtggcggcggagagtcaacgccgccggcccaagcgcgacggccgcggcgcctcgctggtggcgcagccggtgtggtcggcactccggccggcgaggggaccggaacgcggaaggcgtggactgtgtgtggagtaggctgcaacggaaatggctttggaccgaactgcgggcctctttcgatggcacgggccgggttagtctctcgattcagcccattaacatacccacgaatcccaaacccgcacgcctctccgagacgcttgaaaattcaaaatggcgctgctcgcctgtcaaaccctccctacctgtcagc

SEQ ID NO: 20 - protein, 284 aa, Setaria italica
MALLSCETLPVDLTPTPPKLFEIPQQNPSPGNRCLHPPVRRPLLLLCQRGGGMGAEYPTFADVSGARALLFLADSSPAPPPPPPPPALSDEFSCYSGSSSSSSYSGASARSCVSDSARRGRPVDPLRVLSVVASLRRINPKMLAEATGALFHSGAEKKRKGVWIEVDSYEDQSERSSTVASEGSTVTAAASAGSTATSGRCRRPPRASGGGDGGGEKAPRRAEVIMQWFSRSQAGPATENDIRAAVGDNSGTSKAIRWLLKQEGGLRRAGTGGLLDPYVYMVAG

SEQ ID NO: 21 - DNA, 1700 bp, Setaria italica
  source 1..1700: mol_type = other DNA
tgaccatctagcttccgcagcttcactatcttattcttcttttttcgttgacttattcgcaagtgaaaaaactttgtattcttgttaccagcgtaagccacaatattctcgaatggttgatcttttaatatattcatagttagtttactgcatatttggtatttccgatctacattagacagggaggctgcaactatcatctacattagacagcttataatcttttaaacgaggatgaagcgtaattaagaaaacaatataatcattatcgaaattagacagcttataggcttttaaacaaggatgaagcatataagtaagaaaacaacacaaacctcttctgtgagggtttgacagtcgtccctcggttgaagataatgttgcgcattgagtcaatagaatcgcgtacagcaatcacaccaaacacttgcagcggccactgtaagtctgccggactgtttccaactttaaccctgatattataaatctgtagactattggagtggccatcatacacatatcggggcacagatgaatttgagtagtccatggccaggaccattgctgcaggtatttgtcagagatttagattcagaccaattagcatcagcaatggtattcctatactaagatgcatgcattttattttcacaaacatatacatcagcccgcgctagatatacatgctagtagaagtcgctagataggactaagaacacttagaactgaattggcaactgacaaggggcgaaaaatattgatgtataatggagaactcactgatgtcctcgaattccaggccgggattccttgcaatccagtcgtcgcggaacttgagatgatccggggcgtatttgcggtagtacgccaataacttgttgtgcgccttcactcccttttctccagcttcctttgcttgtcatatttttcctcgagttccaccattttcttgcgctttatcagtagcatgatctccataaaaattttcatctcggagcgcttccatcatcgcgctctaattttcttctgctcttcttcctgcttgcgattatcctcctcaactttcttgtgctcttcttcctgctcgcgattatcatcctccttccggtaaaagggagggagcaacccgcgattatcctcctcctccggagtctccttccaaaagagaaggaactcctccgaaagattttccatctcgcacggcgcggacacagctgagttggaggaaaccctagataatgccagcacggcggcaccgctttttatgagaggctgagtggtcgcgtaagcaagggctcgtttggtatggtttacggtagctttagcttcgcaattgtagtagtattgtagtaggtgtagttgaagctgcacggagttcaccagtttcacctccttcacagttcattttaagatttcggtatccgttccagcttcgtgctacagtagcacacacggtgcttaatagtgcgggttactggcgcgtagccgaaacccacctatgcggtgccaaaagggcctaaataggccggactctgaactgtgtggagtaagatgtggaccgaactgcggacctctttcgtgtgcagtaggcctttatccgctcttgatggcatgggccggattaggatctcgattgagcccattaaggcacccaccaatcccaaatccgcacgcctctccaagacgccgaaaaattcaaa

SEQ ID NO: 22 - DNA, 3446 bp, Oryza sativa
  source 1..3446: mol_type = other DNA
atggattcagctcttgccgccgccgccgccaccgccgccgtggcggcgttccccaacttcgccgatgtcgccggcgccgtcgcgctcctcgttctcgccgactcgccgccagcgccgtccccgcctccacctcctcccactgtcaggtactacttccgcgcgcagcactctggttttctccattcggtgttcggcgcgacgaattcaccgccccgtgcggcggcgcctccccgttcattgcagcgacgagctctcgtgctactcgggatcctccgcgtcctactccggcacctcggcgaggtcgtgcgtgtccgactccgcgcagcgcggtcgccccgtcgaccccctccgcgtgctcgccgtcgtcgcctccctccgccgcatcgaccccaaggtaaataatctgatgtgctagatcgcgaacgccaatagtttcatcggtgctaatggctctgctcctcgttgactctaggtgctcgccaaggcgacgaacacgctgtttcagggcgagtcgtcgaagaagaggaagggcgtgtggatccacatcgacgacgatgaggacgagagcgagaggaacagcgctgtggccagcgaggggagcaccgtcacggggaccgcgtctgcaagctccacggccacgtcggggagatcccaccggcctccgcgggcgagtggcggtggcgaccagctgccgcgaagggcggacaagatcatgaagtggttgtcgcggccgggagcggtgccggcaacggagacgaccatccgtgcagccgtcggcgacaatgctggcaccagcaaggcgctgcgcttgtgagtaccacactgctgccaattgcaatttgaaacgcttttgaaatggttgtcatgttaatttcaatttgaagcggttttgaaatggttgccatgttaattccaatctgaagcggttttgaaatggtttccgtgttgctaaattgtttgtgtggaatccgcaggctgctgaagcgtccgggctgcttgcgacgttctggctctggtggccgcaatgatccatatgtttacatggtatgtgaaactcatgcctactgtttgaattgagacgggaatacgaaattcgatgcatgttctgtgctgttgaatgatggtggctcattgtattgtagcttaagagattttcagattggagtagtattaccctttgatagtgtgcaaattttttgaatcgagttagtgagctactgagcttagtaatttccagtggcccagtgggacatacacatttgaagcagttcttaaacctgagttaaatttgcggtagtgctcactgatcactctttagtaccccattctagaagctctatttattgtgttgggagctcagcacaggcaaagttcacagcaacagtgacatgttatgacggccactgcacaggcagacatcacaaaatgtgatgtctcaaatttgtgaattatcagctcttattaactttgcagaatacattcattactatttagctattcccatgatttctcatgagtactattttccttttgatgaattttcaggtcacaggctgacccaatgaagttaactaagttgtgtttatagcaaaagagatggtaactgtaaagctattggagaggccaagtgacttctgagcagtatttagtaatttctactacctgtctgtcttaattactgtttaattttgccttcctgcatataagaagtatagccataggagaaagcaaagttcccatgttagaaagagcttgcagatatatttgtaaagataggctactggatagaaatatagacacacaatcatacacccagagattttatgtcatcgcgctttatattagtgtatgtcgaactggagtaatcaagtataatgagttccattttcagacacttctttttgcaattaactgttatctgaacttctctgtgggaatgaaatctgtgctgttgaataggaattattagatattttgaatagtcccttctgtcagctctttattgtacaaatagcctacggttctgatgaacaaatctgtattgaagttcaatccatttatttctccttctgttgcagaagctagtgaagctaccaatctccacattggttgtttttcacatataactaaagctaaactaacgcgctacgatgagcatacgctagagttagctttgttctgaaatctgacagtggaagcctttgaggctttcacagcttgatccttttgtctgcatactttagttcctcttcttactggagagcatgttcttgaaccagaaagctgagtccttggggtacctctttagcgtcttgtagtccacgtagacgatgccaaaacgggaagtgtacccgagcctccactcgaagttgtcaagcaatgaccaagcaaagtatccaatcactttggcaccatcgtctatcgccttcttgagctcagtgatgtagtttctgtagtatctgattcttactgtatcatgcacaccctgagtgatactgacgttgccaggttggtccatacctgtaagagcacaacattgatcatagtgtaagccttctttaatacttttcattgtcaacaaaactattattcggagaatcagggaccattctgatagaattagtaccattttcagaaaggatcattgtagggtttccatatgtttcctttacataggtcacagccttgttgattccccatggcacaatgtaaagccagtaggagtttgcctgcgtgaattcaatcaattcaactattgaagtgtgatgatttaacttgaaattcaacttttcctaactgttcaacatactactgtacaaatgtacaatacattgactgccacttacttgagctccaatgggcacgccgtttcgttcatctggaaaagacatgagtgcatgagcaactgatccaattaaacagtactacaagaaactgaattatgcatcagaaaatcagaaggatttgggggtggagtattcagaactcacaggcaaacccaacatgccagtcatcctgataactggtcggtgtcaggttccatggcccagggtctttcatgtagaaagaggtgtagtggttgatgccaacataatctattgagtctttcaccatcctggactcctcatcactgaaggttggcatcctgtccttgacgatttcgagcatcgagtatgggtaccgaccatggataatggggtcaaggaacctttaaggagatcatagtgtcagtttgctgatccaaaaaaaagggtactttcagactttcagtttgtggctacactatcacaaaaaatctagactgctactagacctgagcatctgaactctggagtaaataatcgataatttgatcctgaaccagatggtgttttcagatttttaccatccgaggtgaaaatctcttgccctctgtgctgcagccctatcagcattgctgtcactga

SEQ ID NO: 23 - DNA, 1700 bp, Oryza sativa
  source 1..1700: mol_type = other DNA
gccgatcaatctccaatgacatcaaccttggctgccggtaaccaacagagaccagatcttgcagccacgcggacatgaactgcaaatccttctctccccagaacccctcctcagtcatcgcataactcaaatcaagaatacgttcaaagagtgtctgcttatgtgaccgccactccattaggaaatctatcagcctccctacactgacatggatatccttctcatcatcaaacgggtgagcatgcacattatccatgcggtgcacggtcggtgggtagaccactaagtaacccccaatctcccataatatgcgctgcgaccaatacccacgtataacatcagctgccattggactcactgatactggcaatgcaagcccccaaaatgccggtgaatggaacagtgtattaactgagttgattggagccatcatgccctgaggaagtgcgaccttaggcgcatctgcatcaaaccggagatcaaaagcttccatttccgatgatttccgagtgaagtagaacacagcgtcaacatccggtaggccattgcacagcccctgctgtatgaactgcccgccaccgaacacctgagtgtagaattcttccacacccacctccccggctttgtgcagcggcaacccccgcggccacaccgacggctgcccaaagtgcacgtagggattcaccaccgtgcggttgggatccgcatggctgtactgcaagagcacgccgcccccttggcgatggtccagatcgacatcgaagtgcttggtgaggttactgcccaggacggcgttgcgcgcgtccgcgtcgtagatgacgcgcgcgccgcgctgcacggcgaagaggtaggcggcggccttgcgggcgtggccgcgcgcggggaggaaggcaacggagcggaacccgaggcgcgcctggtcggcgagcgtgagcagcgcggcgcccgggtgcgaccagtccgggggcgtggtctcgtcggccacggcgaggagctgccacccggggaccgcgggcagcgggcggtggcgcgggtggtgggccgccgcggcgaagatgatccacctggaggcgcgcagcgacgggagcggcgaggacgggagcaccgggagagggggcacgcgcgaccacgcgacagacgggtactggagccggcgcgacgcaccgccggagcgtgcggcggcgaggcagagcgcggacggggacccgccgccgtagaggaggagcagcaggaacggcgccgccgcgagcgccgcgaggacgacgtaggccacgcggcatctggaactcgccatttttgggcgtagtggtggtggcgagatgggagaggggagaggcgacttggtggagtactactactcctccacttggtagtagtggcgttgctgccgaggtggacagctagtcctcgtcggagtcaatggcctcgcggcggcgccgcgcagcccgcgaggttgtttcgccgccggcgacgaggcccgagaggcgacagagagaggggattttccccattttgggcccaatcatgggcctttatgggccttgatgggccgtgtgtagccgtgcctatcctatgggccgttgctttgctggcttgcacacgtcatcgcgtgctggctttccaacacgctcgaaaattcaaaagttgtctcccttttgatatccccacaaaaatctcgagccccacttgccggcgacgag

SEQ ID NO: 24 - protein, 257 aa, Oryza sativa
MDSALAAAAATAAVAAFPNFADVAGAVALLVLADSPPAPSPPPPPPTVSDELSCYSGSSASYSGTSARSCVSDSAQRGRPVDPLRVLAVVASLRRIDPKVLAKATNTLFQGESSKKRKGVWIHIDDDEDESERNSAVASEGSTVTGTASASSTATSGRSHRPPRASGGGDQLPRRADKIMKWLSRPGAVPATETTIRAAVGDNAGTSKALRLLLKRPGCLRRSGSGGRNDPYVYMIFTIRGENLLPSVLQPYQHCCH

SEQ ID NO: 25 - protein, 229 aa, Oryza sativa
MDSALAAAAATAAVAAFPNFADVAGAVALLVLADSPPAPSPPPPPPTVSDELSCYSGSSASYSGTSARSCVSDSAQRGRPVDPLRVLAVVASLRRIDPKVLAKATNTLFQGESSKKRKGVWIHIDDDEDESERNSAVASEGSTVTGTASAGSTATSGRSHRPPRASGGGDQLPRRADKIMKWLSRPGAVPATETTIRAAVGDNAGTSKALRLLHEASGLLATFWLWWPQ

SEQ ID NO: 26 - DNA, 954 bp, Triticum aestivum
  source 1..954: mol_type = other DNA
ttttagaagcctattagtcaagtcttaattggtaggcttctaacaaaataaatttggttgagcttctagaataaactgggtaggggtagcttatcccaacttattatagaaaccagcaaaagaactggcacttaaaacgtagttcgaggctaaaattacaagatcacgagaaatcgtgaggacaggtaatcgcctcaatatgattccttatcaacatgttctttaaactgattatttcgtagcttaaaatccatacagatgttcctaaatagtagtaaaggccaggaagtccctattcctaggacaacaacattcctagaatcctcaatcctctcccttccgacctgctgctacaacatgccgaccttacaccctctacacgccaataagaactcatggagcttgggtgccaacgatagtcactagtaccattatacagagttactccctccattcataaatataagatattttactatcttttctgaattagatgtatatagacacgttttagtttttcccgcaaaaaagaagaagacacgttttagtttgtttgttcacttatttcagtccgtatgtagtttatattgaaatattcaaacatcttatatttatgaaccgaggggttacattttaagaaggatacttacaaaaatgataggcaaatgaaaaattatgcaacagaaagagttgccccgaatcaaaatccagtaagatctgcagatatcatcctcgacccgtgcggcctggcgagcggcgaccccgcatgtcagccccccttgaacccaaccgcgccgcacgcctccctacacgggcgctacacactttgaaatttcaaaatggcaccgctactgctaccatcctcccccaaaacttctcaattccccaacaaaatccaactccgccactgcctcctcctctcccaccgccacggcctgcccccttgccggcgac

SEQ ID NO: 27 - protein, 236 aa, Triticum aestivum
MGPAPATASRAAAFPTSADVALAGALLCLADSPPAPLLPTLSDELPSYSGSSSSYSVTSARSCVSDAARRSRPIDPLRVLAVIASLRRVDPKVLVQATSKLFQGELAKRRKGVWIEVINGEEEEGGEESERGSVVASEGSTITGPASSRSTATSGRCRRPPLASGGGDALLRRADSIMKWLSRPKAGPATETAIRAAVGDNAVTSKALRWLLKQKRGLRRAGTGGRPDPYVYMIAG

SEQ ID NO: 28 - DNA, 3220 bp, Triticum aestivum
  source 1..3220: mol_type = other DNA
ggacaacaacattcctagaatcctcaatcctctcccttccgacctgctgctacaacatgccgaccttacaccctctacacgccaataagaactcatggagcttgggtgccaacgatagtcactagtaccattatacagagttactccctccattcataaatataagatattttactatcttttctgaattagatgtatatagacacgttttagtttttcccgcaaaaaagaagaagacacgttttagtttgtttgttcacttatttcagtccgtatgtagtttatattgaaatattcaaacatcttatatttatgaaccgaggggttacattttaagaaggatacttacaaaaatgataggcaaatgaaaaattatgcaacagaaagagttgccccgaatcaaaatccagtaagatctgcagatatcatcctcgacccgtgcggcctggcgagcggcgaccccgcatgtcagccccccttgaacccaaccgcgccgcacgcctccctacacgggcgctacacactttgaaatttcaaaatggcaccgctactgctaccatcctcccccaaaacttctcaattccccaacaaaatccctcaattccccaacaaaatccaactccgccactgcctcctcctctcccaccgccacggcctgcccccttgccggcgacatgggcccggcccccgccaccgcctcccgcgccgcggccttccccacctccgccgacgtcgccctcgccggcgcgctgctctgcctcgccgactccccgcccgcgccgctcctccccaccctcaggtaaccgcgaatccgccgtaccgctaactggttttctgcccgcacccgcttccgcgttccgcgacgggcgctcaccgccgcgtgcgcgtctccgcaattgcagcgacgagctcccctcctactcgggctcctcctcctcctactccgtcacctcggcgaggtcgtgcgtgtccgacgcggcgcgccgcagccgccccatcgaccccctccgcgtgctcgccgtcatcgcctccctccgccgcgtcgaccccaaggtaaccaacctcctcgcctgctagttcgcggtttgtccgtcgtgtctgtcttctgcggcgatgcgctgatcgatggttctggtcggtggctctaggtgctggtgcaggcgaccagcaagctgttccagggcgagctggcgaagaggcggaagggagtgtggatcgaggtcatcaacggcgaggaggaggagggcggggaggagagcgagaggggcagcgtggtggccagcgaggggagcaccatcacgggacccgcgtcctcccggtccacggccacgtcggggaggtgccgccgacctccgctggcgagcggtggtggtgacgctctgctgcggagagcggactcgatcatgaagtggctctcacggccgaaggcggggccggccacagagacggccatccgcgccgccgtcggcgacaacgctgtgaccagcaaggctctgcgctggtgagtgggacacttcctccaatccaactgtcgtctgaagatgttctgaaatttgttgcctaactgcccaatctctgtttgttcgtgcgtctctctgttcggaattcgcaggttgctgaagcagaaaagggggttgcgtcgtgcaggcactggtggccgcccggatccgtatgtgtacatggtacgtgaaatttgtgccacacagtttgcgttggaatgggaatgtcagtttcaaaatatactatgtgtgtttggatgatgattgctcaaggtagctgaaaaaaacttgcaatcagtataactcactgtcactatgcaacctttagatgcagttagggagttgttacttattggagtagtaatttccagttacccagtagagagtaagtggaatctgaaaacagttgttaaatctgattaacttatcctagttccactcacagcctcagttctataattctggaagccccattaattgaaccctgaaccctgcaaattttgtggcaacattgatgtctgacgatcaacttggtagcccaggtccttaagtaattattttgaccacattctgagactttctaacttagctgcatgcatactgtacttaccaattcccaggcttctcatgtgtatactaattgtttctacgaattttcagattgcaggctgaagtggtgaagttaagtaagctgtgttatagcgaaggagttggcacctatatatatctgctggaaaggtcaagttagcttttgaagagtatttagtaatttctagcacctgctgtttaatcggtccagttttgcctagccttcccgcaaataacaaatataggataggaatagatatttacgtgttcgcaatagcttgcaaagcactggtgaaaagctcttggatagaaagataaatggatagtcgtatagatggcagtttcatgattacgaagtcattattcatgtgtgtttagaatcagtataatccttgcaagaagtacgagaaataatcggtactgttggaatcagtataataaaatatgatgggttcagttttggggtactggtttgcagtgaattgctcatttggctttcctctgtgaggtcatatgtatgctgttgaaattgtctcatttgatatgatgatattcaattatcccatgtgcttcagtttacctaaactgatctgcttgtggtgttttgactcttgggaaacaagacttagaagacatgtctttgcttcgcattgtattatatataaggacgggcaaagccacaatctcaatggccttttatttcaagtttcttaaagctaagtccgaggttcatttttggggtactggttacattatgattttattctgaaagctggagcaactgatcctcccggatcttgtttgcaatcctagcctctcttcttctctgagagcatgttcttgaaccacaaggctgagtccttggggtacctcttcagagtattgaagtccacatagacgatgccgaaacgggcagtgtagccgagtctccactcgaagttgtcaagcagagaccaagcaaagtacccaaccactcgtgctccattgtctatcgccttcttgagctcggttatgtagtctctgtagaaccggatccttactgtgtcatgtacaccgtcagcgatgctgacatttcctggctggtccattcctgtcagatcataacattgatcattcgtgcccttgttagtgat

SEQ ID NO: 29 - DNA, 1115 bp, Triticum aestivum
  source 1..1115: mol_type = other DNA
aattccccaacaaaatccaactccgccactgcctcctcctctcccaccgccacggcctgcccccttgccggcgacatgggcccggcccccgccaccgcctcccgcgccgcggccttccccacctccgccgacgtcgccctcgccggcgcgctgctctgcctcgccgactccccgcccgcgccgctcctccccaccctcagcgacgagctcccctcctactcgggctcctcctcctcctactccgtcacctcggcgaggtcgtgcgtgtccgacgcggcgcgccgcagccgccccatcgaccccctccgcgtgctcgccgtcatcgcctccctccgccgcgtcgaccccaaggtgctggtgcaggcgaccagcaagctgttccagggcgagctggcgaagaggcggaagggagtgtggatcgaggtcatcaacggcgaggaggaggagggcggggaggagagcgagaggggcagcgtggtggccagcgaggggagcaccatcacgggacccgcgtcctcccggtccacggccacgtcggggaggtgccgccgacctccgctggcgagcggtggtggtgacgctctgctgcggagagcggactcgatcatgaagtggctctcacggccgaaggcggggccggccacagagacggccatccgcgccgccgtcggcgacaacgctgtgaccagcaaggctctgcgctggttgctgaagcagaaaagggggttgcgtcgtgcaggcactggtggccgcccggatccgtatgtgtacatgattgcaggctgaagtggtgaagttaagtaagctgtgttatagcgaaggagttggcacctatatatatctgctggaaaggtcaagttagcttttgaagagtatttagtaatttctagcacctgctgtttaatcggtccagttttgcctagccttcccgcaaataacaaatataggataggaatagatatttacgtgttcgcaatagcttgcaaagcactggtgaaaagctcttggatagaaagataaatggatagtcgtatagatggcagtttcatgattacgaagtcattattcatgtgtgtttagaatcagtataatccttgcaagaagtacgagaaata

SEQ ID NO: 30 - DNA, 27 bp, synthetic construct
  misc_feature 1..27: note = oligonucleotide
  source 1..27: mol_type = other DNA
agaagccaacgccawcgcctcyatttc

SEQ ID NO: 31 - DNA, 51 bp, synthetic construct
  misc_feature 1..51: note = oligonucleotide
  source 1..51: mol_type = other DNA
caagcagaagacggcatacgagatagaagccaacgccawcgcctcyatttc

SEQ ID NO: 32 - DNA, 58 bp, synthetic construct
  misc_feature 1..58: note = oligonucleotide
  source 1..58: mol_type = other DNA
aatgatacggcgaccaccgagatctacactctttccctacacgacgctcttccgatct

SEQ ID NO: 33 - DNA, 82 bp, synthetic construct
  misc_feature 1..82: note = oligonucleotide
  source 1..82: mol_type = other DNA
gtcccccagatttgctaggacaccgccgtacccacaccccggatcagttaatatcgtagacataatggccctccctgccatc

SEQ ID NO: 34 - DNA, 21 bp, synthetic construct
  misc_feature 1..21: note = oligonucleotide
  source 1..21: mol_type = other DNA
tataaacatacggatccaggg

SEQ ID NO: 35 - DNA, 68 bp, synthetic construct
  misc_feature 1..68: note = oligonucleotide
  source 1..68: mol_type = other DNA
catcatgcatgcagcaggctgtgctgtggacctgatcgagtttcaattgatccaagcaagcaagaggg

SEQ ID NO: 36 - DNA, 21 bp, synthetic construct
  misc_feature 1..21: note = oligonucleotide
  source 1..21: mol_type = other DNA
ccctggatgcgtatgtttata

SEQ ID NO: 37 - DNA, 442 bp, synthetic construct
  misc_feature 1..442: note = oligonucleotide
  variation 402: note = n is a, c, g, or t
  source 1..442: mol_type = other DNA
aattgcagagagagaccagtcgttgatcggtcggagatctccccgaatcgatctcgatcggccggaattttgggggccggccgggaggtggatgatggggggtgattaccatctaggcggcgccgtgttggatgatgtaccaacatatcactataggctactttgggaacctcagatccccttcgggattggaggaaattgagatggaaatgaactaatttcttctctaacccccttcaatcacgaaggggattcgagtttccaaactagccctatatatatcagagctagggagggatattgctgcaacatgcatcacgaggtacagatatataattagggttctgctgcaatgttttgactatagatcgttagtgatctgctgtaattaataataatggngatctcgaagcaattctaaacatcatagtagcaggcccag